(19) United States
(12) Patent Application Publication
(10) Pub. No.: US 2025/0248601 A1
(43) Pub. Date: Aug. 7, 2025

(54) EVALUATION OF HEALTH CONDITIONS OF A HUMAN USING THERMAL ANALYSIS OF THE ABREU BRAIN THERMAL TUNNEL

(71) Applicant: Brain Tunnelgenix Technologies Corp., Aventura, FL (US)

(72) Inventor: Marcio Marc ABREU, Aventura, FL (US)

(73) Assignee: Brain Tunnelgenix Technologies Corp., Aventura, FL (US)

(21) Appl. No.: 19/044,274

(22) Filed: Feb. 3, 2025

Related U.S. Application Data
(60) Provisional application No. 63/549,412, filed on Feb. 2, 2024.

(51) Int. Cl. A61B 5/01; A61B 5/00

(52) U.S. Cl. CPC A61B 5/01; A61B 5/0008; A61B 5/6817; A61B 5/742

(57) ABSTRACT

A medical device for monitoring biological parameters through an Abreu Brain Thermal Tunnel (ABTT) is provided. By monitoring and analyzing the temperature of the ABTT terminus, it is possible to diagnose Covid and other infectious diseases based on characteristics of the temperature signal at the ABTT terminus, even in the absence of any other symptoms.

### CROSS-REFERENCE TO RELATED APPLICATIONS

This application claims the benefit of priority to U.S. Provisional Patent Application No. 63/549,412, filed on Feb. 2, 2024, which is hereby incorporated by reference in its entirety.

### TECHNICAL FIELD

This disclosure relates to a medical device for monitoring the thermal signature of a human subject using frequency or spectral analysis of the thermal signature.

### BACKGROUND

Diagnostics for measuring and monitoring an array of biological parameters exist. Among the many biological parameters that can be measured are pulse, blood pressure, heart function (EKG), brain function (EEG), temperature, etc.

### SUMMARY

This disclosure provides a method of identifying Covid in a patient. The method comprises positioning a temperature sensor on the patient's Abreu Brain Thermal Tunnel (ABTT) terminus; continuously reading the temperature output from the ABTT terminus; displaying a graph of the continuously read temperature output on a display; and identifying a characteristic Covid signature from the displayed graph.

Advantages and features of the embodiments of this disclosure will become more apparent from the following detailed description of exemplary embodiments when viewed in conjunction with the accompanying drawings.

### BRIEF DESCRIPTION OF THE DRAWINGS

FIG. 1 shows facial thermal emission from an Abreu Brain Thermal Tunnel (ABTT) terminus, highlighting that the maximum temperature on a human face is at the ABTT terminus.

FIG. 2 shows another view of the human face, highlighting that the maximum thermal emission on the human face is at the ABTT terminus.

FIG. 3 shows a further view of the human face, highlighting that the maximum thermal emission at the ABTT terminus is greater than the thermal emission from the forehead of the human face.

FIG. 4 shows a still further view of the human face, highlighting that the maximum thermal emission at the ABTT terminus is greater than the thermal emission at any other location on the human face, including the forehead.

FIG. 5 shows a view of the superficial temporal artery of the human face.

FIG. 6 shows a perspective view of the thermal emission from the ABTT terminus in comparison to the thermal emission from the human forehead at the location of the superficial temporal artery, and the thermal emission at the ABTT terminus is greater than the thermal emission at the human forehead.

FIG. 7 shows a front view of the human face, highlighting that thermal emission from the ABTT terminus is higher than the thermal emission from any other location on the human face, including the forehead.

FIG. 8 shows a cross-sectional view through the skin of the forehead of the human face, showing a thick, insulating subcutaneous fat layer adjacent to the dermis, which is below the epidermis.

FIG. 9 shows another cross-sectional view through the skin of the forehead of the human face, showing the variability of insulating subcutaneous fat layer adjacent to the dermis, which is below the epidermis.

FIG. 10 shows a cross-sectional view of skin at the ABTT terminus, which is over the ABTT, highlighting that the skin has the thinnest dermis of any skin on the human face and has no subcutaneous fat.

FIG. 11 shows the vascular network in the human face.

FIG. 12 shows the thermal emission from the human face, highlighting that even with the vascular network throughout the human face, thermal emission from the ABTT terminus is markedly greater than any other location on the human face.

FIG. 13 shows a cross-section of skin in the malar region of the human face, showing the thick subcutaneous fat layer adjacent to the thick dermis, which is adjacent to the epidermis.

FIG. 14 shows a view of the human face, highlighting that thermal emission from the horn or arc-shaped ABTT terminus is significantly higher than thermal emission from the human neck.

FIG. 15 shows a cross-sectional view of the skin in the human neck, showing the thick layer of fat, which reduces thermal conductivity significantly, adjacent to the dermis.

FIG. 16 shows the relationship between the frontal vein (FV), the superior palpebral vein (SPV), the angular vein (AV) and the ABTT terminus.

FIG. 17 shows a volume rendering reconstruction of a 1 mm-slice coronal CT scan that shows the relationship between the FV, SPV, AV, and ABTT terminus.

FIG. 18 shows thermal emissions (infrared light emissions) from the left orbital region that shows three distinct, and lower, infrared light emissions from the FV, SPV, and AV as compared to the much higher infrared light emissions from the ABTT terminus.

FIG. 19 shows a view of the human face showing thermal emissions (infrared light emissions) with a measured temperature of 36.8 degrees Celsius (98.2 degrees Fahrenheit).

FIG. 20 shows a view of a human face, comparing the insulating characteristics of subcutaneous fat in the forehead to various types of wood, and showing the relative thickness of the skin at the ABTT terminus, which is thinner than the thickness elsewhere on the human face and includes no subcutaneous fat. This figure also shows that the temperature in the forehead is measured at 37 degrees Celsius (98.6 degrees Fahrenheit), indicating no fever condition, in comparison to a measured temperature of 38.2 degrees Celsius (100.8 degrees Fahrenheit) at the ABTT terminus, indicating a fever condition.

FIG. 21 shows a comparison of the temperature measured with a “corrected” thermometer with a 1-degree Celsius compensation, indicating a forehead temperature of 38.1 degrees Celsius (100.6 degrees Fahrenheit), falsely indicating a fever, in comparison with a measured temperature of 37.3 degrees Celsius (99.1 degrees Fahrenheit) at the ABTT terminus, correctly indicating that there is no fever.

FIG. 22 shows a view of a first human face skin color type.

FIG. 23 shows a view of the thermal emissions of the first human face skin color type, showing the highest emission is at the ABTT terminus.

FIG. 24 shows a view of a second human face skin color type.

FIG. 25 shows a view of the thermal emissions of the second human face skin color type, showing the highest emission is at the ABTT terminus.

FIG. 26 shows a view of a third human face skin color type.

FIG. 27 shows a view of the thermal emissions of the third human face skin color type, showing the highest emission is at the ABTT terminus.

FIG. 28 shows a graph of temperature changes at the ABTT terminus for a first patient in comparison to core temperature during preparation for surgery (#1 and #2 time points), injection of local anesthetic (#3 time point), incision (#4 time point), and subsequent measurements every 3 minutes, indicating that temperatures or thermal emissions at the ABTT terminus correlate positively with pain intensity.

FIG. 29 shows a graph of temperature changes at the ABTT terminus for a first patient in comparison to core temperature during preparation for surgery (#1 and #2 time points), injection of local anesthetic (#3 time point), incision (#4 time point), and subsequent measurements every 3 minutes, indicating that temperatures or thermal emissions at the ABTT terminus correlate positively with pain intensity.

FIG. 30 shows a graph of temperature changes at the ABTT terminus for a second patient in comparison to core temperature during preparation for surgery (#1 and #2 time points), injection of local anesthetic (#2 time point), incision (#3 time point), and subsequent measurements every 3 minutes, indicating that temperatures or thermal emissions at the ABTT terminus correlate positively with pain intensity.

FIG. 31 shows a graph of temperature changes at the ABTT terminus for a fourth patient in comparison to core temperature during preparation for surgery (#1 time points), injection of local anesthetic (#2 time point), incision (#4 time point), and subsequent measurements every 3 minutes, indicating that temperatures or thermal emissions at the ABTT terminus correlate positively with pain intensity.

FIG. 32 shows a graph of temperature data measured at the forehead of the human as compared to temperature data measured at the ABTT terminus, using identical sensors, when placing a hand of the human into cold water, indicating that the hypothalamus anticipates the hypothermic condition indicated by the hand versus the reaction at the forehead, which indicates vasoconstriction.

FIG. 33 shows a graph of the temperature at the ABTT terminus as compared to the temperature at the human's rectum during sleep, indicating that the temperature of the ABTT terminus correlates with specific thermal characteristics of sleep while the rectal temperature remains nearly consistent throughout sleep.

FIG. 34 shows a graph of the temperature at the ABTT terminus as compared to the temperature at a nasal passage, an esophagus, and the forehead of the human during and following an operation, indicating that only the ABTT was able to detect the hypothermic conditions to which the human was subjected during the operation.

FIG. 35 shows graph presenting the temperature at the ABTT terminus as compared to the temperature of a swallowed temperature sensor while jogging on a treadmill in a climate chamber held at 40 degrees Celsius (104 degrees Fahrenheit), indicating the hyperthermic environmental characteristics triggered a hypothalamic response more quickly than the swallowed temperature sensor.

FIG. 36 shows a graph presenting the thermal response at the ABTT terminus when placing the hand of a 34-year-old female volunteer in warm, 40 degrees Celsius (104 degrees Fahrenheit) water for 10 seconds followed by placing the hand in cool, 18 degrees Celsius (64.4 degrees Fahrenheit) water for 10 seconds, and repeating for multiple cycles. The volunteer's core temperature remained unchanged throughout, but the graph shows that the temperature at the ABTT terminus responded dramatically to the alternating temperatures.

FIG. 37 shows a graph showing changes in impulses with the temperature signal at the ABTT terminus, showing that impulse heights are generally consistent except during incipient cooling at around 36 seconds and the initial rise in reaction to the cooling.

FIG. 38 shows a graph of the temperature of the ABTT terminus of a resting volunteer, showing normal fluctuations in ABTT terminus temperature.

FIG. 39 shows a graph of the temperature of the ABTT terminus of the resting volunteer of FIG. 38 in the upper portion, showing normal fluctuations in ABTT terminus temperature, and upper and lower peaks without connecting lines in the lower portion, showing the consistency of height fluctuations of temperature measurements of the ABTT terminus.

FIG. 40 shows a graph of the temperature at the ABTT terminus during the engorgement phase of sexual relations of the human, documenting a preponderance of narrow (less than 2 seconds wide) impulses during this phase.

FIG. 41 shows a graph of the temperature of the ABTT terminus of a female human during post-orgasm phase of sexual relations, indicating greater than or equal to 4 second wide impulses and fluctuations during this phase.

FIG. 42 shows a graph of the temperature of the ABTT terminus after first injection of dye while receiving a tattoo. The temperature increase indicates, similar to previous figures, a degree of pain. The vertical lines are indicative of electronic interference during the tattooing procedure.

FIG. 43 shows a graph showing the temperature of the ABTT terminus during sleep until the human awoke at 9000 seconds.

FIG. 44 shows a graph of the temperature of the ABTT terminus of the human represented by the graph of FIG. 43 for a 100-second window prior to the onset of sleep.

FIG. 45 shows a graph of the temperature of the ABTT terminus of the human represented by the graph of FIG. 43 for a 100-second window prior to the onset of sleep.

FIG. 46 shows a graph of the temperature of the ABTT terminus of the human of FIG. 43 during early delta stage of sleep.

FIG. 47 shows a graph of the temperature of the ABTT terminus of the human of FIG. 43 during late delta stage of sleep.

FIG. 48 shows a graph of the temperature of the ABTT terminus of the human of FIG. 43 during REM stage of sleep.

FIG. 49 shows the graph of FIG. 48 with the data points indicated.

FIG. 50 shows a graph of the temperature of the ABTT terminus of the human of FIG. 43 during stage 2 (S2) sleep.

FIG. 51 shows a graph of the temperature of the ABTT terminus of the human of FIG. 43 during a 100 second window of awakening.

FIG. 52 shows a graph of an ultra-low frequency power spectrum generated by spectral domain analysis of the temperature of the ABTT terminus during stage 2 and REM sleep.

FIG. 53 shows a graph of the temperature of the ABTT terminus during room cooling from 26.18 degrees Celsius (79.12 degrees Fahrenheit) to 25.76 degrees Celsius (78.37 degrees Fahrenheit), a 0.42 degree Celsius decrease, showing a corresponding rise of 0.11 degrees Celsius at the ABTT terminus, from 35.83 degrees Celsius (96.49 degrees Fahrenheit) to 35.94 degrees Celsius (96.69 degrees Fahrenheit), indicating compensation of room cooling by the hypothalamus over an interval from 300 seconds to 1,100 seconds.

FIG. 54 shows the portion of the graph of FIG. 53 from 500 seconds to 600 seconds.

FIG. 55 shows a graph of the temperature of the ABTT terminus during a first series of successive room warmings.

FIG. 56 shows a detail of the graph of FIG. 55 from 1,050 seconds to 1,100 seconds.

FIG. 57 shows a detail of the graph of FIG. 55 from 1,130 seconds to 1,210 seconds.

FIG. 58 shows a detail of the graph of FIG. 58 from 1,365 seconds to 1,445 seconds.

FIG. 59 shows a graph of the temperature of the ABTT terminus during a second series of successive room warmings for an interval from 7,000 to 8,500 seconds, using the same human measured during the first series of successive room warmings.

FIG. 60 shows a detail of the graph of FIG. 59 during the interval from 7,640 seconds to 7,740 seconds.

FIG. 61 shows a detail of the graph of FIG. 59 during the interval from 8,125 seconds to 8,275 seconds.

FIG. 62 shows a graph of the temperature of the ABTT terminus during a third series of room warmings using a different human from the first and second series of room warmings from 3,100 seconds to 3,400 seconds.

FIG. 63 shows a detail of the graph of FIG. 62 during the interval 3,330 seconds to 3,350 seconds.

FIG. 64 shows a first graph of the temperature of the ABTT terminus of a patient who is in a pre-febrile-2 condition with an incipient stage of influenza who became symptomatic two days later.

FIG. 65 shows a graph of an 800-second window encompassing a trough in the temperature of the ABTT terminus of the pre-febrile-2 human.

FIG. 66 shows a detail of the graph of FIG. 65 from 12,400 seconds to 12,500 seconds, indicating serial shifts of the baseline and accompanying pulses suggestive of cytokine-induced alteration of the temperature of the hypothalamus of the pre-febrile-2 human.

FIG. 67 shows a graph of the ABTT terminus temperature of the pre-febrile-2 human indicating a plateau during a 2,000-second interval.

FIG. 68 shows a detail of the graph of FIG. 67 from 16,000 seconds to 16,200 seconds showing oscillatory behavior of the ABTT terminus of the pre-febrile-2 human.

FIG. 69 shows a graph of the ABTT terminus temperature during an interval that appeared grossly flat, but magnified the oscillatory behavior of the pre-febrile-2 human is apparent.

FIG. 70 shows a detail of the graph of FIG. 69 during the interval from 18,550 to 18,650 seconds.

FIG. 71 shows a graph of the ABTT terminus temperature of the pre-febrile-2 human during a downward slope, showing the oscillatory behavior of the ABTT terminus temperature.

FIG. 72 shows a detail of the graph of FIG. 71 during the interval from 24,520 to 24,620 seconds.

FIG. 73 shows a graph of the ABTT terminus temperature of the pre-febrile-2 human during an upward slope, showing the oscillatory behavior of the ABTT terminus temperature.

FIG. 74 shows a frequency analysis of the temperature of the ABTT terminus of the pre-febrile-2 human, plotting power versus frequency during a 500-second flat phase.

FIG. 75 shows another frequency analysis of the temperature of the ABTT terminus of the pre-febrile-2 human, plotting power versus frequency during a 500-second flat phase.

FIG. 76 shows a graph of a second of two periods of the temperature of the ABTT terminus of the pre-febrile-2 human.

FIG. 77 shows a detail of the graph of FIG. 76 from 43,100 seconds to 43,900 seconds, at the beginning and end of cytokine-induced temperature rise.

FIG. 78 shows a detail of the graph of FIG. 76 from 43,275 seconds to 43,375 seconds, showing the beginning of cytokine-induced temperature rise.

FIG. 79 shows a detail of the graph of FIG. 76 from 43,800 seconds to 43,900 seconds, showing the end of the cytokine-induced temperature rise.

FIG. 80 shows a graph of the temperature of the ABTT terminus of the pre-febrile-2 human showing recurrent dominance of cytokines on the temperature at the ABTT terminus.

FIG. 81 shows a graph of the temperature of the ABTT terminus of the pre-febrile-2 human, showing a major decline at 63,450 seconds after being dominated by sympathetic behavior.

FIG. 82 shows a detail of the graph of FIG. 81 from 63,300 seconds to 63,400 seconds, showing sympathetic activity.

FIG. 83 shows a detail of the graph of FIG. 81 from 63,400 seconds to right before the major decline, showing a gradual decline before the major temperature drop.

FIG. 84 shows a graph of the temperature of the ABTT terminus, showing a cytokine-induced shift persistent from about 66,750 seconds to about 67,600 seconds.

FIG. 85 shows a detail of the graph of FIG. 84 from 67,000 seconds to 67,100 seconds, showing details of the cytokine-induced shift.

FIG. 86 shows a first brief interval of temperature at the ABTT terminus of a pre-febrile-1 human with an incipient state of influenza, which manifest one day after this graph was obtained.

FIG. 87 shows a second brief interval of temperature at the ABTT terminus of a pre-febrile-1 human with an incipient state of influenza, which manifest one day after this graph was obtained.

FIG. 88 shows a third brief interval of temperature at the ABTT terminus of a pre-febrile-1 human with an incipient state of influenza, which manifest one day after this graph was obtained.

FIG. 89 shows a fourth brief interval of temperature at the ABTT terminus of a pre-febrile-1 human with an incipient state of influenza, which manifest one day after this graph was obtained.

FIG. 90 shows a fifth brief interval of temperature at the ABTT terminus of a pre-febrile-1 human with an incipient state of influenza, which manifest one day after this graph was obtained.

FIG. 91 shows a graph of a sleeping human in a pre-febrile-3 condition while sleeping, showing sympathetic surge and parasympathetic inhibition attributable to cytokines allowing sympathetic dominance in route to fever.

FIG. 92 shows an apparatus for measuring infrared emissions and/or the temperature of the ABTT terminus.

### DETAILED DESCRIPTION

The present disclosure provides a medical device for monitoring biological parameters through an Abreu Brain Thermal Tunnel (ABTT) 12, which was previously described as a brain temperature tunnel, and which is described in more detail in U.S. Pat. Nos. 7,187,960, 8,172,459, 8,328,420, 8,721,562, 8,834,020, and 8,849,389, incorporated herein by reference in their entirety. Since the Applicant discovered the ABTT, the Applicant has further experimented with the ABTT and determined that the measurement of the thermal signatures of the ABTT provides a unique window into many human health conditions. Further, Applicant has determined that this newly identified and characterized Brain Thermal Tunnel is part of a complex thermodynamic system and includes, by way of illustration, an intra-brain thermodynamic subsystem, a brain-heart thermodynamic subsystem, a brain-hormonal thermodynamic subsystem, and brain-environment subsystem, all of which are objects of the present disclosure.

### General Discussion of the ABTT

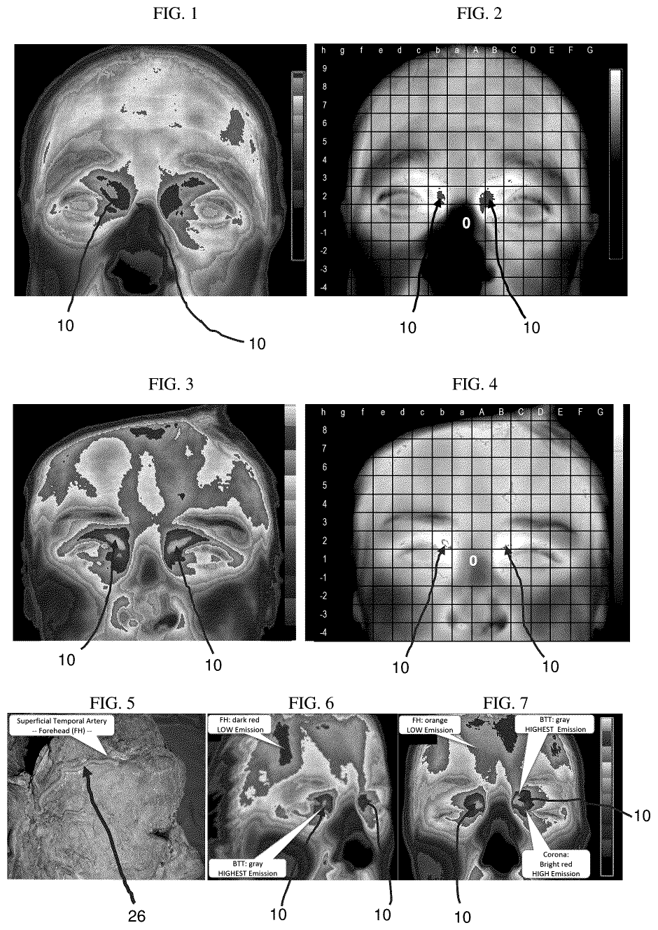

ABTT 12, terminus 10 of which is shown, for example, in FIGS. 1-4, 6, and 7, comprises a continuous, direct, and undisturbed connection between a thermal energy source within a human brain and an external point on the facial skin at the end of the tunnel. The physical and physiological events at one end of the tunnel are reproduced at the opposite end. ABTT 12 allows direct thermal energy transfer through the tunnel without interference by heat-absorbing elements. The source of the thermal heat in the brain is the region of the brain that is a control center for involuntary functions of the body. More specifically, ABTT 12 terminates adjacent the hypothalamus.

The recipient of the thermal heat is four veins that converge to an ABTT 12 “target area” or “terminus” 10, which is at the facial end of ABTT 12. ABTT target area 10 measures about 11 mm in diameter, measured from the medial corner of the eye at the medial canthal tendon and the lacrimal or tear puctum and extending superiorly for about 6 mm, and then extending into the upper eyelid in a horn-like projection for another 22 mm. Applicant recognized that blood flow in ABTT 12 is minimal or stagnant, and, in contrast with other portions of the circulatory system, is bi-directional. Furthermore, Applicant recognized that temperature in the area of the hypothalamus was, contrary to conditions in other portions of the body where temperature is measured, constantly varying. Applicant also recognized that the area of the brain around the hypothalamus has specialized thermodynamics. Still further, Applicant determined that the variation in thermal status presented substantial potential for monitoring the condition of a person because of the speed of temperature variation was indicative of the performance and condition of the body. However, considering that the potential for ABTT 12 is presently unappreciated, understanding the diagnostic capabilities of the output of ABTT target area 10 is currently in the early states.

ABTT 12 is located in a crowded anatomic area. Therefore, the positioning of an apparatus to gather data from ABTT 12 requires special geometry for direct contact with ABTT target area 10 and for optimal thermal transfer, and for non-contact capturing of thermal energy from the area. Referring to FIGS. 16-18 four facial veins converge at ABTT target area 10: frontal 16, superior palpebral 18, supraorbital (not labeled), and angular/facial 20. Angular/facial vein 20 extends from ABTT target area 10, running alongside nose 22, and then extending toward cheek 24; the superior palpebral vein extends from ABTT target area 10 to run along the eyebrow; and the frontal and supraorbital veins extend from ABTT target area 10 to run upwardly across the forehead. ABTT target area 10 is the only location where four veins converge, connecting the center of the brain, i.e., the hypothalamus, to the skin. Additionally, ABTT target area 10 has special vasculature and is the only skin area in which a direct branch of the cerebral vasculature is superficially located and covered by thin skin without or in the absence of a fat layer. The main trunk of the terminal branch of the superior ophthalmic vein is located right at ABTT target area 10 and just above the medial canthal tendon supplied by the medial palpebral artery and supra-orbital vein. ABTT target area 10 on the skin, supplied by a terminal and superficial blood vessel ending in a particular area without fat and void of thermoregulatory arteriovenous shunts, provides a superficial source of undisturbed biological signals including brain temperature, heart rate, blood pressure, blood flow, oxygen levels and oxygen saturation, and body chemistry such as glucose level, and the like, besides carbon dioxide and other gases.

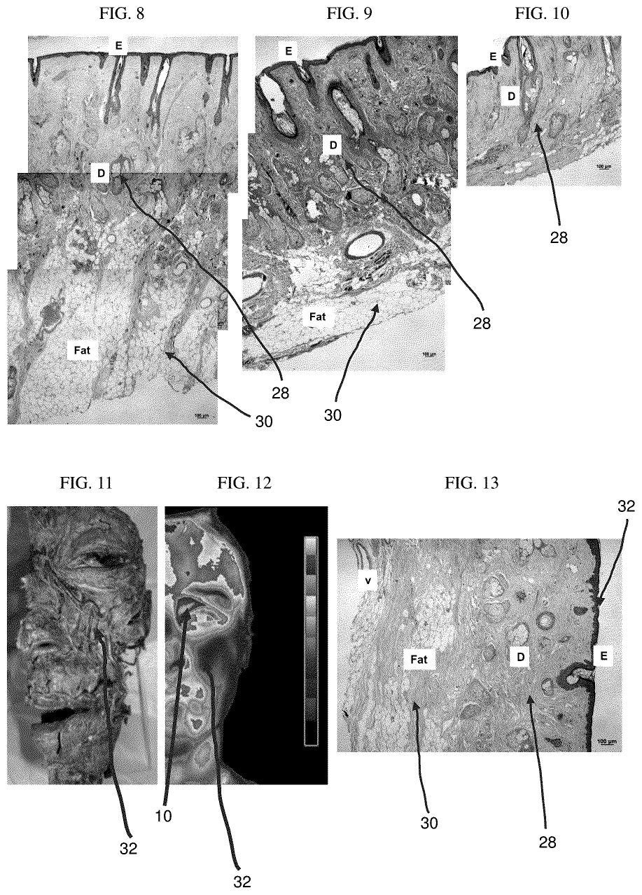

Referring to FIGS. 5 and 8-13, Applicant initially compared the anatomy and thermal physiology between ABTT target area 10 and forehead 14 for several reasons. More specifically because of the latter's proximity to ABTT target area 10 in continuum with the frontal branch of the superior ophthalmic vein as it emerges from ABTT target area 10, the presence of a large superficial temporal artery 26, and the widespread use of forehead 14 for temperature monitoring. Despite its prominent vasculature, forehead 14 reveals low and variable thermal emission as compared to the highest IR light emission at ABTT target area 10. Comparative histomorphometry and proximate superficial anatomy of forehead 14 showed thick and variable layers of dermis 28 and subcutaneous fat 30. See, for example, FIGS. 8 and 9.

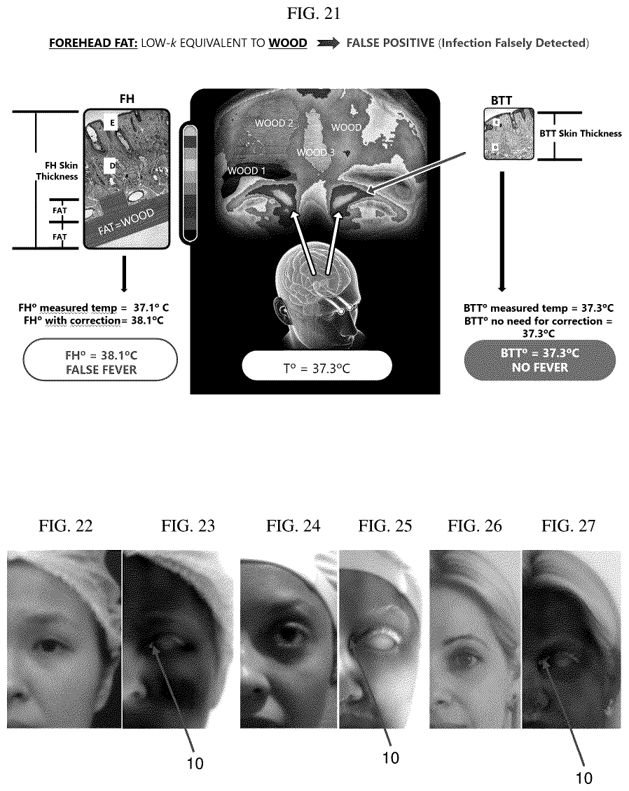

Subcutaneous fat 30 has a low-k, a constant describing the conductivity of subcutaneous fat 30, that is comparable to oak, creating thereby a barrier for transmission of thermal energy in forehead 14 due to thick layers of insulation. In contrast, referring to, for example, FIG. 10, ABTT target area 10 histology shows a fat-free thin skin that lacks customary capillary networks. Variable and high insulation coupled with inconsistent vasomotor tone leads to confounding temperature measurements on forehead 14 (and large superficial temporal artery 26), which has been evidenced by many previous studies, showing the inability of acquiring reliable measurements from forehead 14. As underscored here, forehead 14 thermometry violates laws of physics and biology by trying to measure temperature in an area that is covered by thick and variable layers of insulation. These measurements may, and often do, result in a series of inaccurate results: false negative when true fever is present but is not detected and false positive when fever is falsely detected but the person is truly afebrile. See, for example, FIGS. 20 and 21. Hence, forehead measurements should be abandoned during the COVID-19 pandemic when clinically useful assessments of the body's thermal status are necessary.

The hampered thermal transmission caused by the low-k barrier, characterized by fat and thick dermis and large amounts of capillaries, has led to the inclusion of correction and “fudge” factors in forehead 14 thermometry and thermometry in other body sites. This variable and low thermal conductivity of the body surface leads to confounding temperature measurements during thermal emissive and thermometric forehead scanning, as reported here and by previous reports (noted above). Potentially misleading temperature readings in adults not only impact the group that is most likely to travel but also the most susceptible to COVID-19 (most notably mature adults). In addition, the reliability of the forehead and other facial sites is hindered by its surface vasculature. As shown in FIGS. 5 and 11-13, the superficial temporal artery 26 and other facial vessels are parallel to the skin surface and thus are vulnerable to changes in ambient temperature. Also, their elastic wall makes their caliber prone to changes, not only due to environmental temperature but also to emotional state.

In contrast to forehead 14, the eyelid surface at ABTT target area 10 has consistent thin, fat-free, high-k skin, as shown in specimens of different subjects. For example, FIG. 10, which leads to maximized high thermal conductivity of skin at the ABTT site, which is consistent among different individuals, as necessary in the COVID-19 pandemic. Moreover, measurements at ABTT target area 10 enables unique thermal transmission and accurate and precise temperature measurements that are not influenced by ambient temperature or vasomotor tone (since ABTT 12 is shielded inside the orbit and the vein lacks external elastic lamina thereby preventing any meaningful changes in volume).

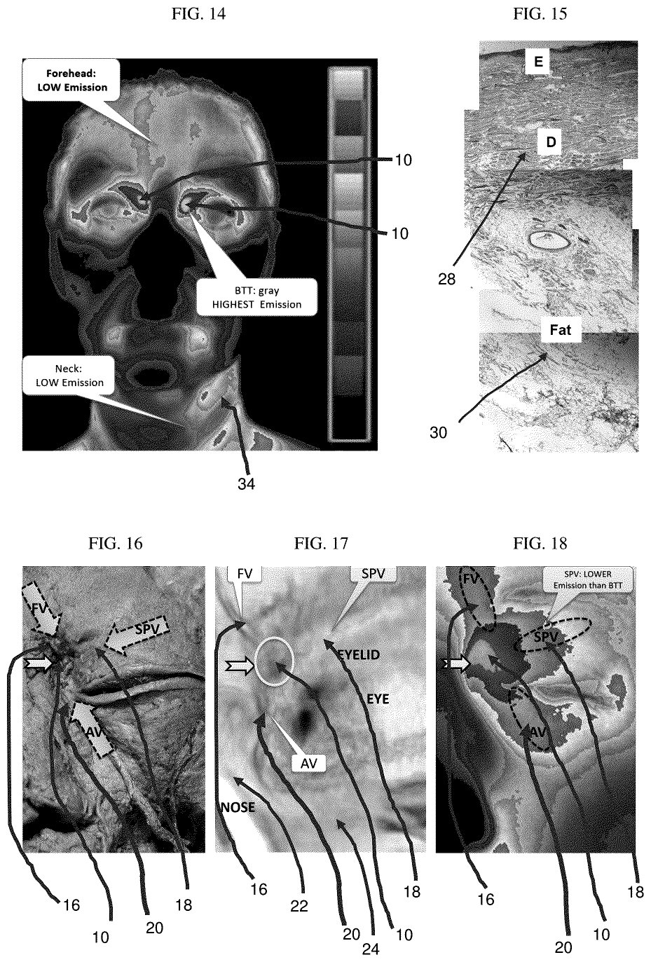

Likewise, despite having a rich and large vascular network, for example as shown in FIGS. 11 and 16-18, malar region 32 has low thermal emission, attributable to low-k histology (thick dermis 28 and fat layers 30) and lack of an underlying tunnel (delineated for ABTT 12). Referring to FIGS. 14 and 15, this pattern is repeated in neck 34, which, despite having major vessels such as jugular vein and carotid artery, also has low-k histology and no underlying tunnel. Hence, neck 34 and the face, e.g., malar region 32, as well as forehead 14 failed to achieve thermal emission comparable to the eyelid at ABTT target area 10.

An irrefutable thermal emissive evidence of the inability of forehead 14 measurements to accurately detect infection and prevent spread of COVID-19 is disclosed exemplarily in thermal emission of FIG. 14. As clearly seen, forehead 14 is very cold and has a temperature approximately 3° C. lower than ABTT target area 10. Low thermal emission observed reflects temperature of a local cold tissue as opposed to the true internal warm status of the body shown at ABTT target area 10. If forehead 14 measurements were taken, they would deceptively indicate normothermia (or hypothermia) and would fail to detect fever in infected populations with SARS-COV-2, both at the individual level or during mass screening at airports and ports of entry. These false negative readings may lead to virus spreading, besides increased morbidity and mortality due to delay seeking care. Despite correction factors, these surface sites, outside ABTT target area 10, may fail to provide thermal emission and temperature measurements that detect the presence of fever, as evidence by even 3° C. difference between the true thermal status of the body and forehead temperature.

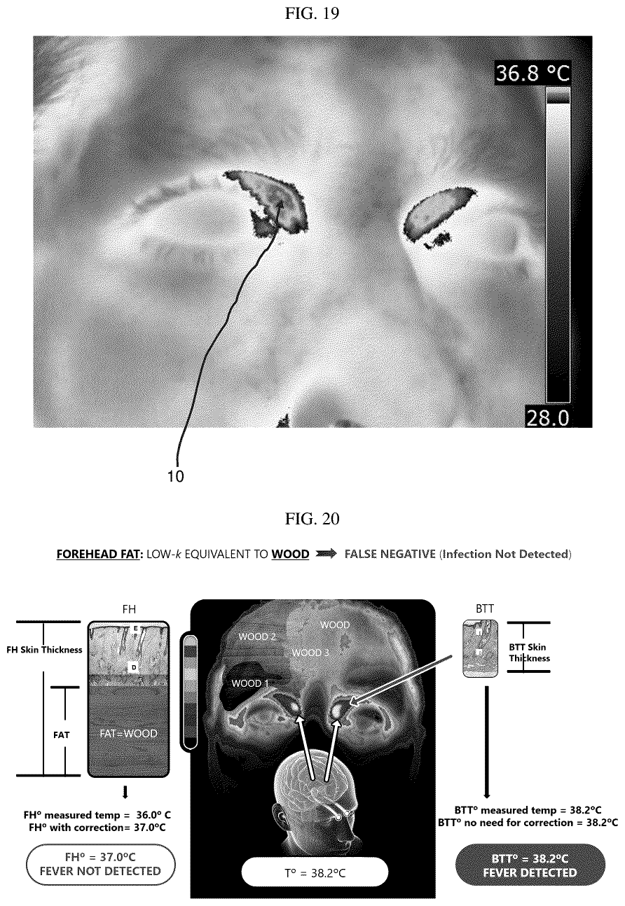

Other figures are presented showing the difference between the emission of ABTT target area 10 and other portions of the human face. For example, FIG. 7 shows a front view of the human face, highlighting that thermal emission from ABTT target area 10 is higher than the thermal emission from any other location on the human face, including forehead 14. FIG. 19 shows a view of the human face showing thermal emissions (infrared light emissions) with a measured temperature of 36.8 degrees Celsius (98.2 degrees Fahrenheit).

The emission of infrared radiation and, consequently, the corresponding temperature of ABTT target area 10 is independent of skin type. FIGS. 22-27 show thermal emissions from human faces having several skin colors. In each case, the infrared/thermal emission from ABTT target area 10 is the highest thermal emission/temperature of the human face, indicating that skin color has little effect on emission from ABTT target area 10.

### Temperature Sensor

All temperature and power graphs presented in this disclosure are from temperatures measured at ABTT target area 10. Multiple configurations of temperature measurement devices can be used as long as those devices meet the requirements described herein. Measurement devices can be contact or non-contact. Contact devices can include thermocouples, high-speed thermometers, and other devices configured to measure temperature. Non-contact devices can include radiometers or infrared sensors configured to receive infrared light emitted from ABTT target area 10 and to convert the emitted light into an equivalent temperature. One exemplary temperature measurement device is described in detail below.

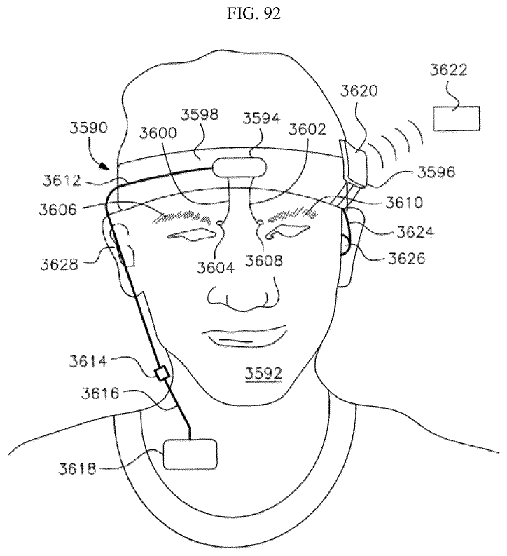

FIG. 92 shows a frontal perspective view of a sensing modular headband 3590 of this disclosure when worn by a user 3592 and including two different biologic parameter modules, namely a BTT temperature module 3594 and an ear monitoring module 3596, said modules 3594 and 3596 including any temperature sensors such as infrared radiation and thermistors.

Given the requirements for temperature sensing to achieve the results described herein, the speed of response of the selected temperature sensor should preferably be comparable to heartrate, since ABTT target area 10 sees temperatures changes comparable to heartrate. Accordingly, a preferably sensor temperature response speed should be 10 seconds or faster. However, given that some characteristics are easier to identify and analyze as higher speeds, 5 seconds or faster is more preferable. Even more preferable is 1 second or faster. Still more preferable is a speed that is the inverse of twice the pulse rate. So if the pulse rate is 80, the speed of response is preferably about 0.01 seconds to obtain the highest resolution temperature data.

BTT temperature module 3594 is disposed on the surface 3598 of sensing modular headband 3590 and includes adjustably positionable arms 3600, 3602 and measuring portion 3604, 3608 positioned below and adjacent to the eyebrow 3606, 3610, and further including wire 3612 which exits headband 3590 and run behind the ear 3628 terminating in connector 3614 which connects to wire 3616, said wire 3616 being connected to a display and interface 3618. Ear monitoring module 3596 includes a wireless transmitter 3620 wirelessly connected to receiver and display 3622, and further including wire 3624 which terminates in ear probe 3626. Each of display and interface 3618 and display 3622 can include a frequency or spectrum analyzer to perform the frequency/spectral analyses disclosed herein. In addition, each of display and interface 3618 and display 3622 is configured to display all the graphs and data presented herein.

### Temperature Data

To assess the anticipated relationship between ABTT target area 10 temperature and brain thermogenesis near its internal terminus, the inventor monitored right (Rt) and left (Lt) ABTT target area 10 temperature and core temperature [sublingual (SL)] prior to, during and after induction of anesthesia (methohexital, rocuronium) and electroconvulsive (ECT)-induced seizure in 47 consenting patients. Per the psychiatrist's clinical decision, stimulation was unilateral (right-sided) or bilateral. ABTT target area 10 temperature not only captured the thermal energy representation of exaggerated cerebral neural metabolism generated by electrical stimulation, but more so the differential responses of right and left hemispheres and their distinction from core temperature.

As seen in Table 1, two minutes after seizure induced by right-sided stimulation (n=24), the temperatures at the Rt and Lt ABTT target area 10 increased by 0.31±0.2° C. (p<0.001 by paired t-test) and 0.17±0.1° C. (p<0.001) above what their respective values had been two minutes after induction of anesthesia (immediately prior to stimulation). This increase is consistent with greater seizure activity on the stimulated side.

Alternatively, in the patients who underwent bilateral stimulation (n=23), the increases in temperature at the Rt and Lt ABTT target area 10 were 0.24±0.2° C. (p<0.001) and 0.31±0.1° C. (p<0.001). Right- or left-sided dominance was not retrievable from redacted data; the inventor thus could confirm that the greater increase in the left side likely was attributable to it more commonly being the dominant hemisphere. Core temperature [sublingual (SL)] remained within 0.01° C. of pre-seizure values, consistent with the specificity of BTT° for brain temperature.

TABLE 3
Temperature Changes in Response to Induced Seizures

| | Core° | Rt BTT° | Lt BTT° |
|---|---|---|---|
| RIGHT-SIDED | | | |
| STIMULATION | | | |
| 2 min after induction of | 36.37 ± 0.4 | 36.38 ± 0.4 | 36.3 ± 0.4 |
| anesthesia | | | |
| 2 min after end of seizure | 36.38 ± 0.4 | 36.69 ± 0.3 | 36.55 ± 0.4 |
| Δ | 0.013 ± 0.05 | 0.31 ± 0.2 | 0.17 ± 0.1 |
| BILATERAL | | | |
| STIMULATION | | | |
| 2 min after induction of | 36.37 ± 0.4 | 36.31 ± 0.3 | 36.37 ± 0.3 |
| anesthesia | | | |
| 2 min after end of seizure | 36.38 ± 0.4 | 36.55 ± 0.4 | 36.68 ± 0.3 |
| Δ | 0.013 ± 0.03 | 0.24 ± 0.2 | 0.31 ± 0.1 |

Confirmation of selectivity of ABTT target area 10 for hypothalamic-pituitary axis activity was assessed as to whether, in contrast to core temperatures, measure for example at the rectum, internally, or skin other the ABTT target area 10, ABTT target area 10 may uniquely capture pain and its intensity attributable to signals from pain-related circuitry in the hypothalamus.

Here the inventor assessed ABTT target area 10 in response to acute pain, a stimulus shown by positron emission tomography (PET scan) to likewise activate the hypothalamus. The inventor monitored patients (n=20) undergoing surgical procedures after infiltration with local anesthetic (but no systemic analgesia or sedation, so that they can leave promptly after surgery) with serial measurements. Baseline ABTT target area 10, core [sublingual (SL)], and surface [forehead 14 scan (Fhscan)] were recorded 15 min prior to entering the operating room. In the operating room, ABTT target area 10 and core were measured continuously; Fhscan and visual analogue scale (VAS) pain scores (0-10) were measured upon injection and at subsequent 3-min intervals or upon new onset of pain. Devices were compared with paired t-test. The data are summarized in Table 2 below.

With VAS=0, pre-operative ABTT target area 10 temperature was 0.005° C.±0.07 lower than SL temperature (P=NS) and 0.01° C.±0.06 lower than FHscan temperature (p=NS). Upon injection, for which patients reported an increase of mean VAS 0-10 pain score from 0 at baseline to 4.75±0.8, ABTT target area 10 temperature increased from 36.58±0.2° C. to 36.87±0.2° C. (Δ0.29° C.) (p<0.001). Alternatively, mean SL temperature and Fhscan temperature remained at their common baseline of 36.58±0.2° C., with ABTT target area 10 temperature—SL temperature being 0.30±0.13° C. (p<0.001) and ABTT target area 10—Fhscan temperature being 0.31±0.14° C. (p<0.001). Post-injection pain scores varied among patients: subjects with max VAS scores 0-2, 3-5 and 6-8 had ABTT target area 10 differences of 0.23° C., 0.32° C., and 0.43° C. from preoperative baseline.

TABLE 2
BTT°, SL°, FH° and VAS pain scores during local anesthetic injection and incisional pain.

| | | | | Max When Max | Max When Max | Max When Max |
|---|---|---|---|---|---|---|
| | Preop | PreInjection | Injection | Subseq Pain 0-2 | Subseq Pain 3-5 | Subseq Pain 6-10 |
| VAS | 0 | 0 | 4.75 ± 0.79 | 2 ± 0 | 4.11 ± 0.2 | 6.26 ± 0.74 |
| BTT | 36.575 ± 0.19 | 36.585 ± 0.19 | 36.87 ± 0.21 | 36.80 ± 0 | 36.89 ± 0.25 | 37.0 ± 0.19 |
| SL | 36.58 ± 0.21 | 36.58 ± 0.21 | 36.575 ± 0.21 | 36.65 ± 0.21 | 36.57 ± 0.22 | 36.58 ± 0.20 |
| FH | 36.585 ± 0.21 | 36.575 ± 0.20 | 36.565 ± 0.20 | 36.6 ± 0.14 | 36.51 ± 0.22 | 36.63 ± 0.21 |

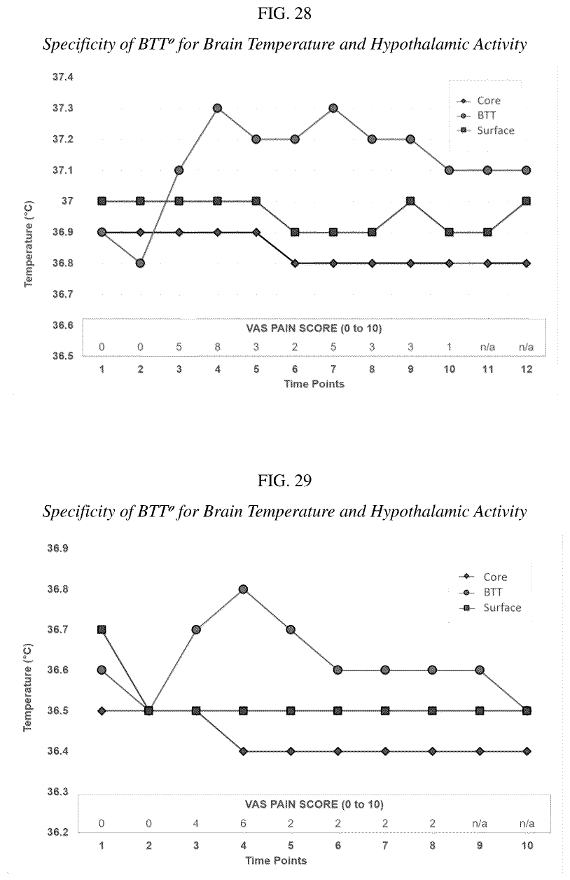

FIGS. 28-31 show graphs of temperature changes at the ABTT terminus in comparison to core temperature during preparation for surgery, indicating that temperatures or thermal emissions at the ABTT terminus correlate positively with pain intensity. Here, the remarkably consistent increase in ABTT target area 10 temperature in response to hypothalamic activation (during injection and subsequent incision and reinjection, if there was subsequent pain due to insufficient analgesia) is shown for representative patients. These data clearly document the lack of detection of HP Axis activity by core temperature (SL) and surface temperature (FHscan®), whereas ABTT target area 10 not only detected hypothalamic-pituitary (HP) axis activity, but also the discriminant level of activity (reflected by ABTT target area 10 temperature detecting the intensity of pain). ABTT target area 10 temperature captured peri-hypothalamic temperature within seconds of the painful stimulus, again indicative of HP axis activation and thermodynamic activation being proportional to an increase in pain. This increase occurred without a change in core temperature or comparable changes in Fhscan temperature, thereby confirming unique assessment of pain activity via the ABTT target area 10 temperature. Hence, ABTT target area 10 temperature again identified an acute response suggestive of hypothalamic activation (via neural pathways) without an accompanying rise in core temperature as monitored by SL temperature or surface temperature by forehead scanning. The remarkable results document the unique capability of high-resolution BTT sensing systems to measure even one of the most abstract brain signals, i.e., pain.

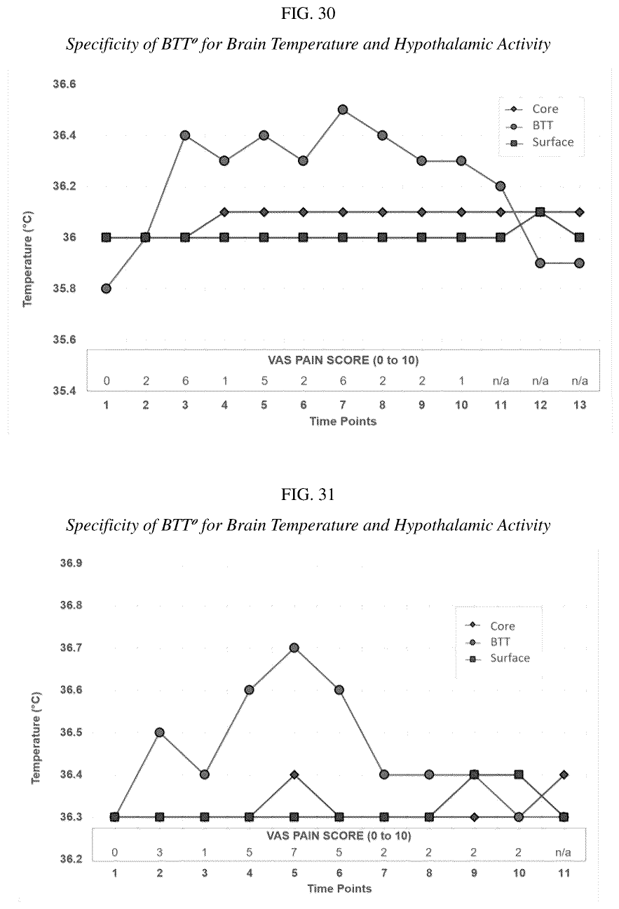

It should be noted that FIG. 28 shows a graph of temperature changes at the ABTT terminus for a first patient in comparison to core temperature during preparation for surgery (#1 and #2 time points), injection of local anesthetic (#3 time point), incision (#4 time point), and subsequent measurements every 3 minutes, indicating that temperatures or thermal emissions at the ABTT terminus correlate positively with pain intensity. FIG. 29 shows a graph of temperature changes at the ABTT terminus for a first patient in comparison to core temperature during preparation for surgery (#1 and #2 time points), injection of local anesthetic (#3 time point), incision (#4 time point), and subsequent measurements every 3 minutes, indicating that temperatures or thermal emissions at the ABTT terminus correlate positively with pain intensity. FIG. 30 shows a graph of temperature changes at the ABTT terminus for a second patient in comparison to core temperature during preparation for surgery (#1 and #2 time points), injection of local anesthetic (#2 time point), incision (#3 time point), and subsequent measurements every 3 minutes, indicating that temperatures or thermal emissions at the ABTT terminus correlate positively with pain intensity. FIG. 31 shows a graph of temperature changes at the ABTT terminus for a fourth patient in comparison to core temperature during preparation for surgery (#1 time points), injection of local anesthetic (#2 time point), incision (#4 time point), and subsequent measurements every 3 minutes, indicating that temperatures or thermal emissions at the ABTT terminus correlate positively with pain intensity.

The inventor further assessed what appeared to be sensitivity of ABTT target area 10 temperature to activation of the hypothalamus with a series of cold challenges akin to those shown by PET scan to cause a hypothalamic response to maintain core temperature during changes in temperature and of associated hypothalamic pathways that regulate heat loss behaviors.

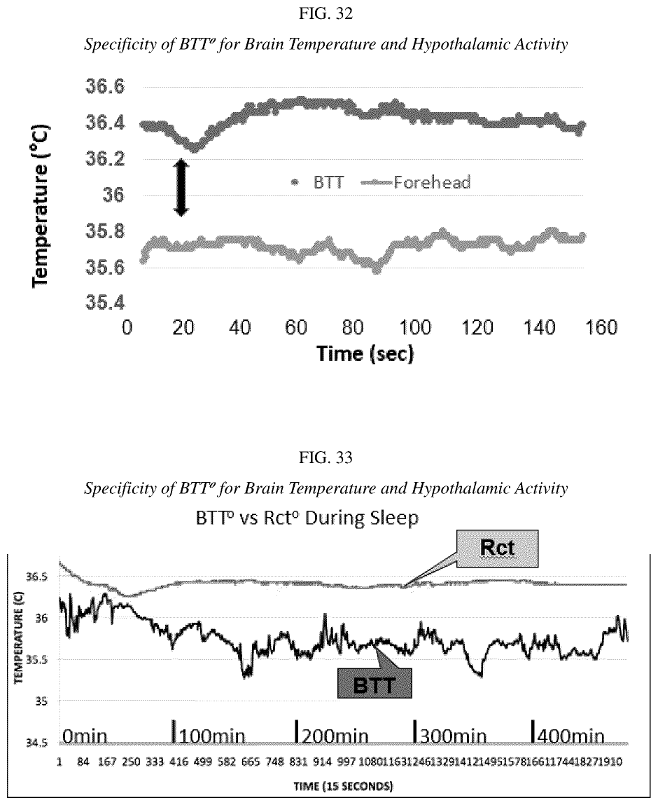

During forearm and hand immersion in 5° C. water for 2 minutes, the inventor monitored a 25-year-old healthy male volunteer member with identical thermal FH temperature and ABTT target area 10 sensors. The cold-water immersion caused ABTT target area 10 temperature to increase from a nadir of 36.25° C. at the onset to 36.53° C., which was of a magnitude comparable to that seen during ABTT target area 10 temperature/core temperature discordance during a challenging mental effort and also pain. In contrast, FH temperature underwent varying decline from 35.75° C. to a minimum of 35.6° C. See FIG. 32. Finger temperature (not shown) declined from baseline of 35.6° C. to 34.0° C. Consistent with these findings, digitalization of thermal imaging performed during bilateral immersion of both forearms and hands in ice water documented temperature at the ABTT target area 10 that increased by 0.54° C. while forehead 14 temperature decreased by 0.52° C.

Referring to FIG. 33, the inventor also compared changes in ABTT target area 10 temperature and core temperature during sleep, as this comparison would represent the integrated impact of autonomic and thermoregulatory neuroendocrine (including Circadian) changes at the hypothalamus. A healthy 28-year-old male volunteer member of the inventor's research team was monitored from sleep onset until awakening with a sensor placed noninvasively at ABTT target area 10 and an invasive equally calibrated thermal probe inserted (10 cm) in the rectum (Rct°). Simultaneous recordings of ABTT target area 10 temperature and rectal temperature) (Rct°) were obtained and are shown in FIG. 33. At baseline, core temperature measured invasively with the rectal probe was higher than ABTT target area 10 temperature and rectal temperature remained at a higher level than ABTT target area 10 temperature.

Most revealing in the graph were the minimal thermal changes and homogeneous geometry recorded by the rectal probe reflecting thermodynamics consistent with a thermal storage area, and thus not reflecting the dynamic status of an organ like the brain (hypothalamus). In sharp contrast, recording by ABTT target area 10 temperature captured the characteristic activity of a highly dynamic organ further supporting that ABTT target area 10 temperature measures hypothalamic thermodynamics. At sleep onset, ABTT target area 10 temperature declined more rapidly and to a greater depth than rectal temperature Rct° (1.28° C. and 0.67° C., respectively) consistent with preferential cooling of the brain being an integral component of sleep and, furthermore, that the preoptic hypothalamus is integral to that process. ABTT target area 10 temperature vs. rectal temperature difference persisted upon awakening, albeit less dramatic (respective increases of 0.88° C. and 0.41° C.), consistent with overall body awakening and increased myothermogenesis. During brief arousal (n=2 episodes), ABTT target area 10 temperature increased by 0.64±0.06° C. while rectal temperature Rct° increased by only 0.08±0.002° C., clearly demonstrating the selective sensitivity of ABTT target area 10 temperature to increased peri-hypothalamic activity.

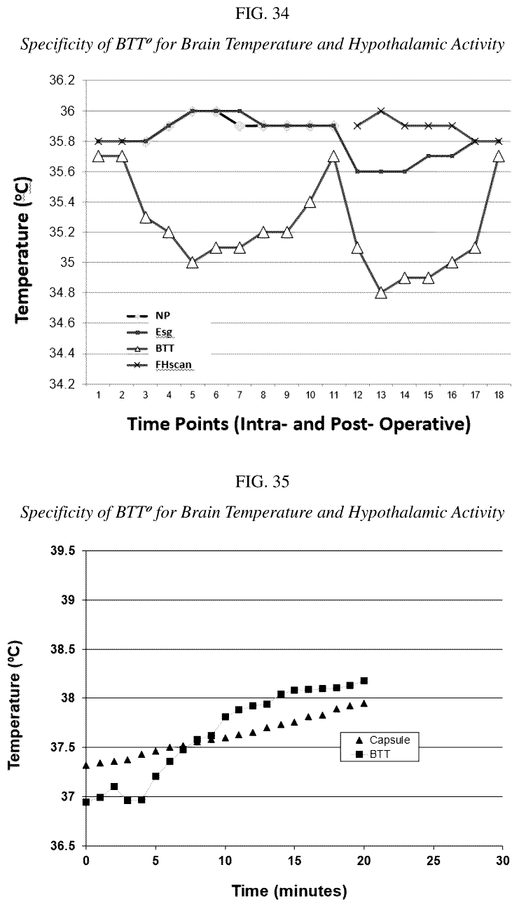

As shown in FIG. 34, the inventor confirmed the discordance between hypothalamic and core temperature in a series of comparative measurements during general anesthesia, which uniquely provides a hypothermic setting in a relatively “silent” brain thermodynamic background thus allowing more precise differentiation of hypothalamic activity. Simultaneous measurement of ABTT target area 10 temperature and nasopharyngeal (NP) temperature (NP°) (representing cephalic measurements in an attempt to measure brain temperature), as well as esophageal temperature (Eso°) (representing standard body core temperature), using equally calibrated thermal sensors are representatively shown here in two surgeries under general anesthesia. As seen in FIG. 34, after induction of general anesthesia, hypothermia ensued and ABTT target area 10 temperature dropped 0.7° C. while, within the same period, invasive measures of the internal temperature of the body showed an opposite response; that is, both NP° and Eso° increased by 0.2° C. ABTT target area 10 temperature was responsive to the onset of anesthesia and hypothalamic depression that ensued, resulting in a 1.0° C. discordance between the hypothalamus and body core temperature (Point 5 in FIG. 34). As general anesthesia was lightened (Point 10 in FIG. 34), ABTT target area 10 temperature increased by 0.5° C., which further confirmed temperature at ABTT target area 10 capturing hypothalamic activation.

Notably, this variation in temperature was not detected by any of the two measures of core temperature (Eso° and NP°). Alignment of ABTT target area 10 temperature to hypothalamic activity was again evidenced as ABTT target area 10 temperature peaked at extubation (Point 11 in FIG. 34) and then returned to baseline (35.7° C.) while core temperature remained unchanged at 35.9° C. In the post-anesthesia care unit (Points 12 to 18 in FIG. 34), ABTT target area 10 temperature uniquely detected hypothermia and showed a decrease in temperature, which returned to baseline levels the following day. These temperature changes also were not detected by either measure of core temperature. Detection of hypothermia during general anesthesia further confirmed ABTT target area 10 temperature sensing hypothalamic depression as captured during body pigmentation (Section IV). As expected, forehead scanning thermometry (FHScan) failed to detect any of the brain (hypothalamic) temperature changes (nor any body core temperature changes). This contrast reinforces in another setting (general anesthesia) that the wood-like low-k structure of forehead 14 prevents acquiring important and clinically useful measurements in the context of the COVID-19 pandemic.

The transition from BTT°/Core° compatibility to divergence such as that seen with respect to fever likewise was demonstrated in the 37° C.-38° C. range during exercise-induced hyperthermia. The graph of FIG. 35 documents observed changes in ABTT target area 10 temperature (measured noninvasively) and core temperature (measured via ingested thermal capsule,) Capsule® in a 28-year-old female volunteer member running on a treadmill in a climate chamber with an ambient temperature of 40° C. Even though both temperature metrics continued to rise, there was a disproportionate increase in ABTT target area 10 temperature at approximately 37.5° C., where the velocity of thermal increase was ˜0.17° C.·100 s⁻¹ for ABTT target area 10 temperature vs ˜0.07° C.·100 s⁻¹ for Capsule®. By the end of the exercise session, there was a relative increase in ABTT target area 10 temperature of approximately 0.5° C., which is similar to 0.56° C. measure for patients with a fever. The inventor attributes this similarity to ABTT target area 10 temperature sensitivity to hypothalamic activation, by mechanisms not dependent on antigen exposure, but that relied on heat absorption and production occurring in this exercise setting, which increased hypothalamic autonomic activity necessary to maintain thermal homeostasis that was clinically evidenced by sweating. ABTT target area 10 temperature and Capsule® subsequently rose in concert. It should be noted that initially the temperature of the gastrointestinal tract being measured by the ingestible thermometer is higher than ABTT target area 10 temperature, consistent with the inventor's observations during sleep studies showing baseline rectal temperature higher than ABTT target area 10 temperature°. However, subsequent divergent thermal changes reveal the hypothalamic heat smyce from the sensor at the ABTT target area 10 in both settings: during sleep ABTT target area 10 temperature continued to decrease as a result of decreased neuronal activity, whereas during exercise the temperature increased with high velocity indicative of hypothalamic activity. After the pronounced increase in ABTT target area 10 temperature (BTT°), the BTT°-Capsule® differences were consistent with arterial and venous temperature differences reported in subjects with thermocouples placed (invasively) in the aorta and an internal jugular vein, where average brain temperature during exercise was calculated to be at least 0.2° C. higher than that of core. However, according to the studies shown here and to the best of the inventor's knowledge in reviewing scientific literature, the thermodynamics observed and acute rise in the vicinity of 37.5° C. is unique to the monitoring of hypothalamic activity by ABTT target area 10 temperature. The autonomic thermodynamic characterization during exercise provides another region in the hypothalamic autonomic thermodynamic spectrum, enabling identifying parameters for hypothalamic programmed fever, discussed further herein, and further distinguishing signal for asymptomatic infection from noise (other regions of the spectrum).

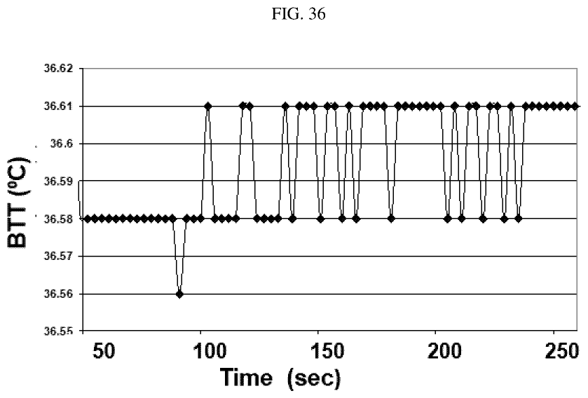

Turning now to FIG. 36, the inventor's findings in the context of thermal challenges delineate the basis for these confounding impacts. Responses during hand and arm immersion in cold water and exposure to decreased room temperature identified Psymp modulation of the impact of these sympathomimetic challenges. Moreover, as noted above, exposure to increased peripheral temperature, i.e., room temperature, and initial hand immersion in warm water induces seemingly unbridled activation of Psymp anti-hyperthermic mechanisms which the inventor believes would substantially compromise an inflammatory response. If the hypothalamus interprets the local inflammatory responses to be manifestations of imminent hyperthermia, then, rather than allowing an unbridled sympatho-immunologic response to destroy the virus, it could bridle the response, thereby deferring an effective response until after the horse (or better, “herd” of viruses) is out of the barn (multiplying at highly infectious and potentially highly injurious rate within the cells).

In addition to the potential for Psymp activation to promote stealth of the virus, it may compromise health of the host, most notably by a virus with a long incubation period. The inventor's findings suggest that herein introduced normothermic analysis of ABTT target area 10 temperature can effectively address these confounders. Although a disturbance may be suggested by measurements of mean ABTT target area 10 temperature and its overall variability, this disturbance initially may be imperceptible, is not specific for infection, and does not identify the components of cyto-autonomic thermodynamics. Alternatively, viral invasion can be detected, characterized, and potentially diagnosed by monitoring cyto-autonomic thermodynamics in accordance with the inventor's herein proposed criteria which, in addition to delineating Psymp activity, also focus on the presence of cytokine-induced thermo-autonomic and thermo-immunologic shifts. From a therapeutic standpoint, delineation of cyto-autonomic thermodynamics may guide interventions that safely promote killing of virus while it is limited in number and not well-sequestered. It may also enable one to deliberately alter the cyto-autonomic thermoregulatory activity so as to activate a desirable sympatho-immunologic response, perhaps by challenges akin to those shown herein (e.g., room cooling, hand immersion in cold water). It similarly may enable indications for and timing of other interventions, including antivirals, administration of antibodies, vaccinations and of medications which activate (e.g., liposaccharides) or alter (e.g., hydroxychloroquine, interferon) the immunologic response, as addressed below. Alternatively, monitoring cyto-autonomic activity also may enable intervention when the inflammatory response becomes excessive (e.g., cytokine storm), as was shown to occur upon exposure of vaccinated animals (against SARS-Cov) to virus, and upon re-exposure after recovery from dengue fever, and that may occur in response to high viral loads.

Thus, the inventor has developed a test that identifies a disturbance that appears to be specific for infection(s). The patterns delineated in the pre-febrile subjects analyzed herein were clearly distinct from those at rest, during sleep, pre- and post-orgasm, pre- and post-dye injection for a tattoo, stressful mental effort, immersion of hand and arm in cold water, and exposure to room cooling and warming. Findings on different days confirmed that the pattern seen in pre-febrile-2 at approximately 48 hours prior to symptoms was attributable to the incipient infection and not a feature of the given subject: tracings from the same subject on different days at rest and during the given subject's participation in studies of room cooling and room warming lacked such a pattern. The findings also distinguished the difference between a pattern one day before symptomatic influenza and >2 days prior to symptomatic influenza, thereby delineating the progress of disease.

The inventor believes assessment of continuous ABTT target area 10 temperature monitoring and more specifically cyto-autonomic assessment of the ABTT target area 10 temperature signal can be introduced immediately to identify individuals with a pattern suggestive of cyto-autonomic disturbance (viewing in the spectral- and/or time-domains as in examples shown herein). Unless the findings can be dismissed based on questioning or evidence of confounding ongoing activity (highly unlikely, thus far the inventor has found none), this diagnostics should prompt infectious precautions pending precise diagnostic viral nucleic acid testing. Moreover, as discussed below, in view of the dependence of a virus on host cells for survival and proliferation, early quarantining and appropriate distancing may not only prevent multi-host viral spread but also community virus survival. Hence, benefits can be achieved immediately for the patient and community. As presented in the discussion, the signatures delineated with cyto-autonomic thermodynamics that result in Brain Autonomic Thermodynamic Signature of Asymptomatic viral Infection (BATS_{AI}) may provide a biophysical (nonchemical) brain-based diagnostics that may replace the need for invasive and uncomfortable swabs, or invasive blood testing, with results being immediately available to patient and doctor to promptly administer any needed therapy to preserve heath and prevent death.

Concurrently caregivers, providers, and investigators, as well as governments, health organizations, and industry can be identifying specific cyto-autonomic profiles. The inventor believes, consistent with the report that immune-HPA axis interactions appear to be virus and phase specific, assessment of cyto-autonomic thermodynamics will provide thermal profiles and even signatures of COVID-19 and other infectious as well as noninfectious disorders; however, one first needs to obtain a database to define a given disorder (e.g., COVID-19) and multiple databases to ensure specificity for the given disorder. Much of this can be obtained during initial implementation.

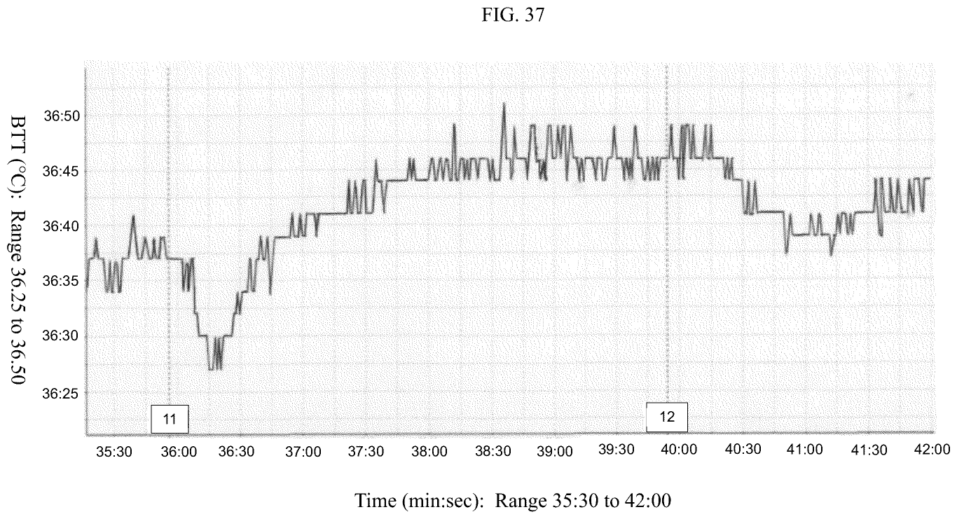

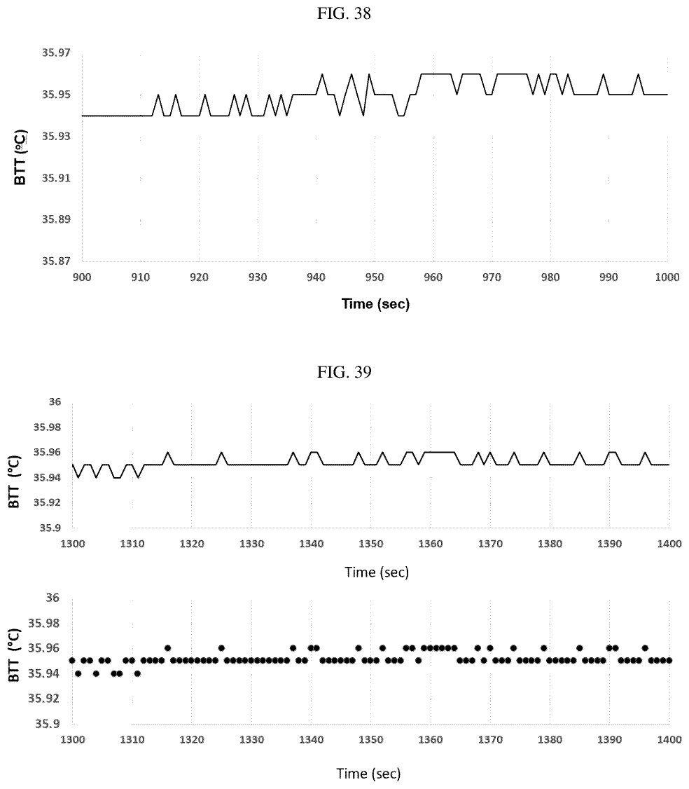

FIG. 37 shows a graph showing changes in impulses with the temperature signal at the ABTT terminus during rest, mental stress, and sleep. ABTT target area 10 temperature graphs obtained in healthy resting subjects, FIGS. 38 and 39 had B/N ratios within the ranges of dominance encompassed by sexual relations and tattoo, as did the pre-challenge period prior to mental effort shown in FIG. 37. The inventor appreciated that at-rest periods are not quiescent; the interaction of Psymp and Symp is dynamic akin to the inconsistencies of heart rate variability where oscillations of healthy heart are constantly changing. The inventor anticipated that, like heart rate variability, ABTT target area 10 temperature impulse patterns are more meaningful in the context of challenge or disturbance.

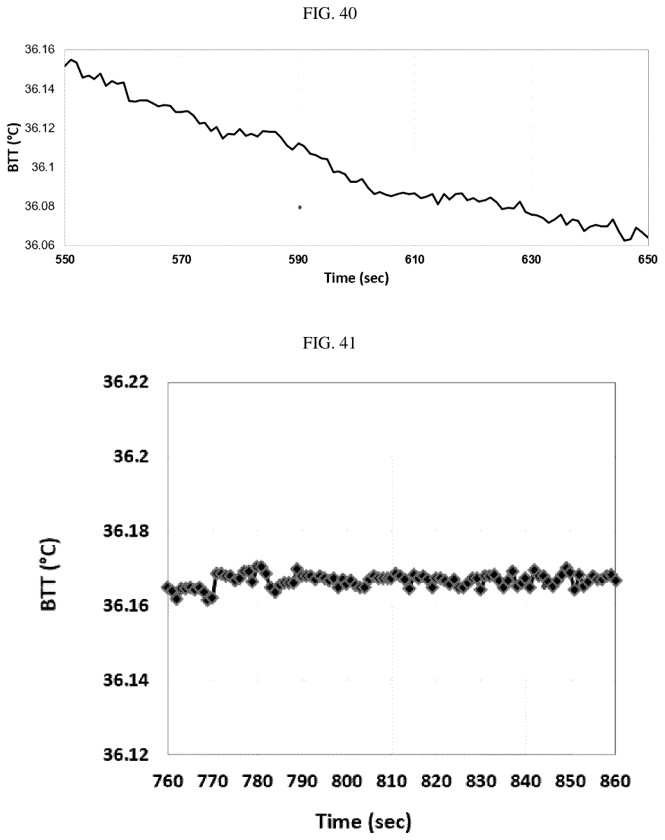

FIG. 40 shows a graph of the temperature at the ABTT terminus in a 100-second window during the engorgement phase of sexual relations of a male subject, documenting a preponderance of narrow (≤2 seconds wide) impulses during this predominantly Psymp phase.

FIG. 41 shows a graph of the temperature of the ABTT terminus of a female human during post-orgasm phase of sexual relations, indicating greater than or equal to 4 second wide impulses and fluctuations during this phase. Preliminary criteria for signal analysis, which are modeled after those established for analysis of cardiovascular signals (albeit with different signal widths and frequencies) were established during the Psymp-dominant engorgement and Symp-dominant post-orgasm phases of sexual relations. Those assessments showed that 1) the incidence and occupancy times for narrow (<2 sec wide) impulses of ABTT target area 10 temperature were greater during the Psymp dominance of engorgement than during post-orgasm; 2) the incidence and occupancy times for broad (4 to 9 sec wide impulses as well as >10 sec wide fluctuations) components of ABTT target area 10 temperature were less during engorgement than the Symp-dominance of post-orgasm; and 3) the Broad/Narrow ratios for incidence and occupancy were greater during post-orgasm than engorgement. The presence of fluctuations (>10 sec) post-orgasm indicated the presence of chemical (e.g., neuroendocrine) inducers of hypothalamic thermogenesis.

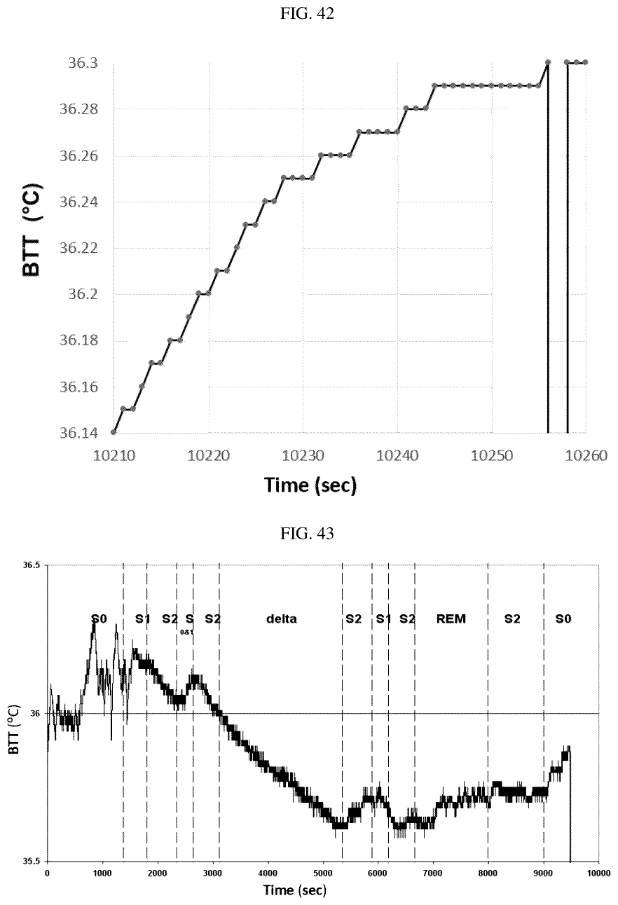

FIG. 42 shows a graph of the temperature of the ABTT terminus after first injection of dye while receiving a tattoo. The temperature increase indicates, similar to previous figures, a degree of pain. The vertical lines are indicative of electronic interference during the tattooing procedure.

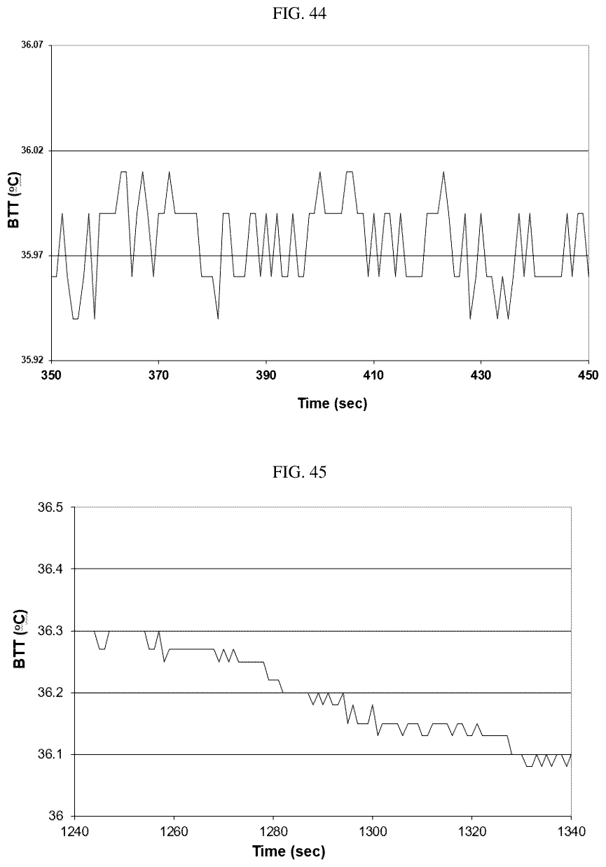

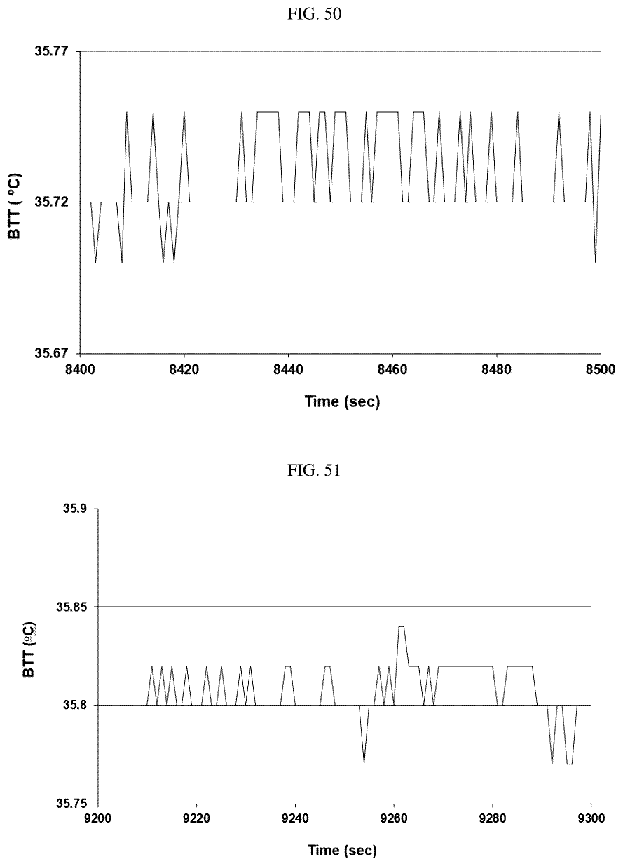

FIG. 43 shows a graph showing the temperature of the ABTT terminus during sleep until the human awoke at 9000 seconds. Assessment as to whether ABTT target area 10 temperature identifies Psymp and Symp impulses and fluctuations during sleep was achieved in a volunteer in whom ABTT target area 10 temperature was continuously recorded in concert with EEG determination of the stages of sleep in successive 30 second epochs in FIG. 43. Findings are displayed in FIGS. 44-51 as 100 sec intervals during pre-sleep, initial sleep, early delta, late delta, REM, stage 2 and awakening.

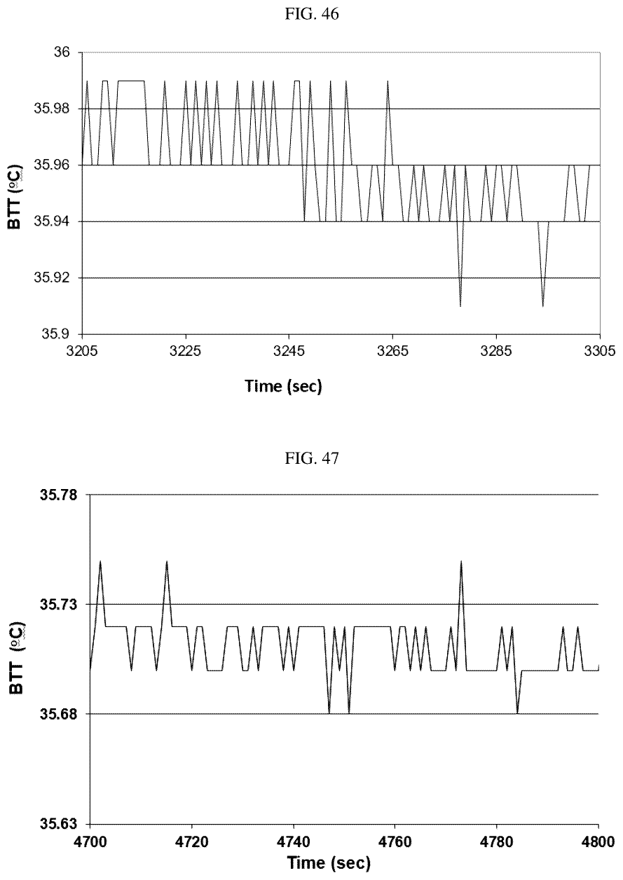

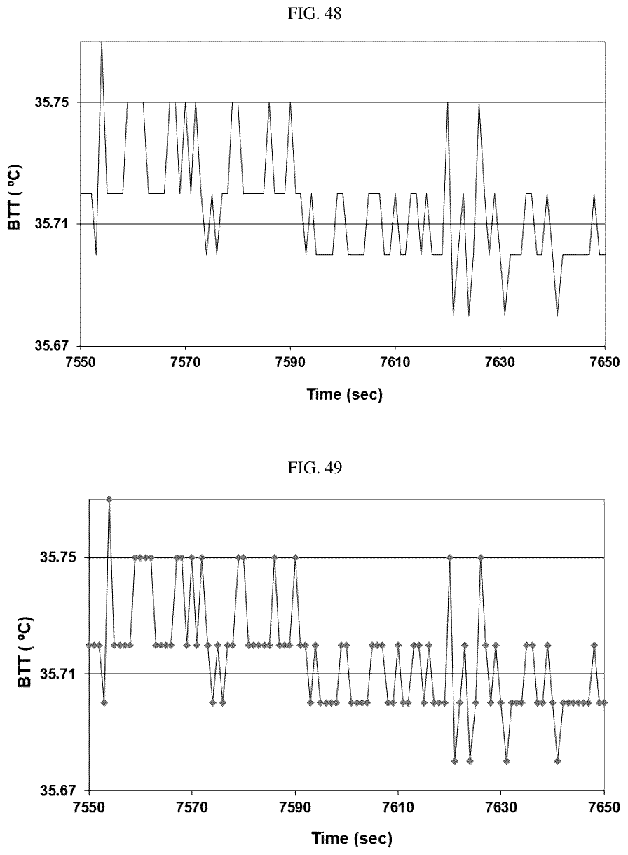

Prior to sleep onset, although mean temperature did not change, Psymp predominance was suggested by B/N incidence and occupancy ratios of 3/20 and 18/40. Consistent with brain depression secondary to Circadian variation, including increased melatonin levels, beginning at ˜2600 sec there was a progressive decline in ABTT target area 10 temperature in the context of Psymp predominance: B/N for incidence and occupancy 3/15 and 12/30 as shown in FIG. 6N. This decline continued during early delta, where the respective ratios were 2/23 and 14/46, as shown in FIG. 46. Psymp predominance abated near the end of delta (7/10 and 42/20), as mean° continued to decrease, as shown in FIG. 47. The increase in Symp was dramatic, suggesting homeostatic Symp activation to prevent further decline in brain temperature. The B/N ratios of the subsequent phases, REM, Stage 2, and awakening shown in FIGS. 48-51, were less dramatic. The persistence of imp&fluc activity during sleep was consistent with animal studies which have suggested well-sustained neurally mediated oscillatory activity during sleep: dynamic network activation of synchronously active hypothalamic neurons has been documented. Neural oscillations, including sleep spindle frequency, occur in association with changes in brain temperature.

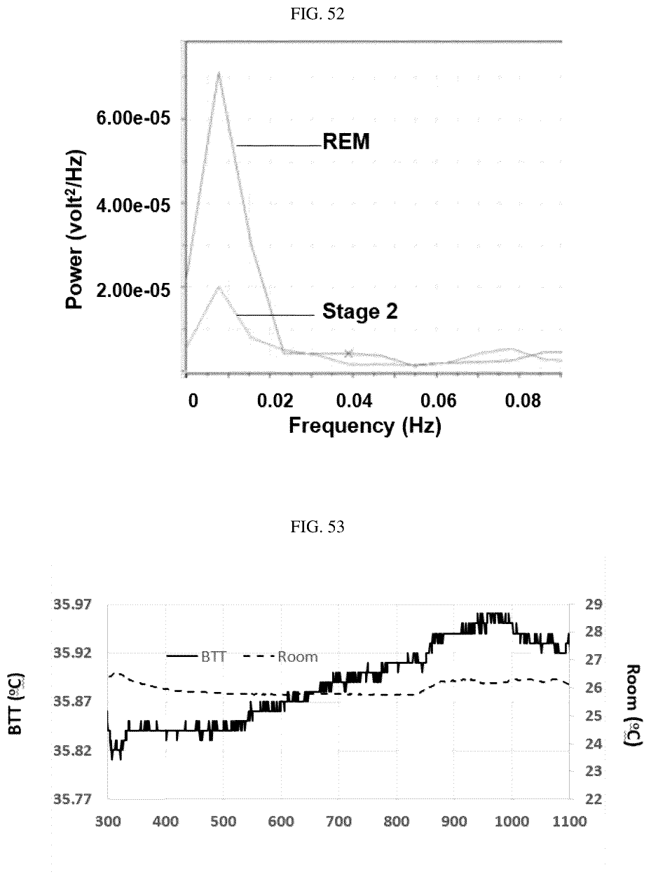

The inventor assessed whether REM caused changes which distort Pysm/Symp assessment. FIG. 52 shows the differing spectral patterns, most notably at the thermoregulatory frequencies, of stage 2 and REM. With the undulation caused by REM excluded from totals, REM and neighboring stage 2 had similar total occupancies (71% and 64%, respectively) and similar degrees of Psymp activity. The ABTT target area 10 temperature findings at the thermoregulatory frequencies are consistent with a report that selective neurons of the hypothalamus are synchronously active during REM sleep. The predominance of power in thermoregulatory range is consistent with the “larger fluctuations” associated with epochs of REM sleep in mice and the “infra-slow” oscillations that co-modulate temperature, heart rate, and brain oscillations during murine sleep. It is not surprising that such an inherent frequency during REM is consistent with the ˜0.01 Hz frequency of the “thermoregulatory band” in cardiovascular waveforms such as that for heart rate variability and evidence of fluctuations of BOLD in this frequency range during functional MRI. Heretofore, there had been an irony with respect to nomenclature: oscillations at ˜0.01-0.03 Hz were said to be within the “thermoregulatory band,” a nomenclature based on cardiovascular signals (variations of heart rate, blood pressure and/or peripheral flow) presumably associated with hypothalamic thermoregulatory activity. However, the corresponding variability of temperature, most notably at the thermoregulatory center, the smyce of the cardiovascular changes, had not been recorded (until now) in undisturbed humans.

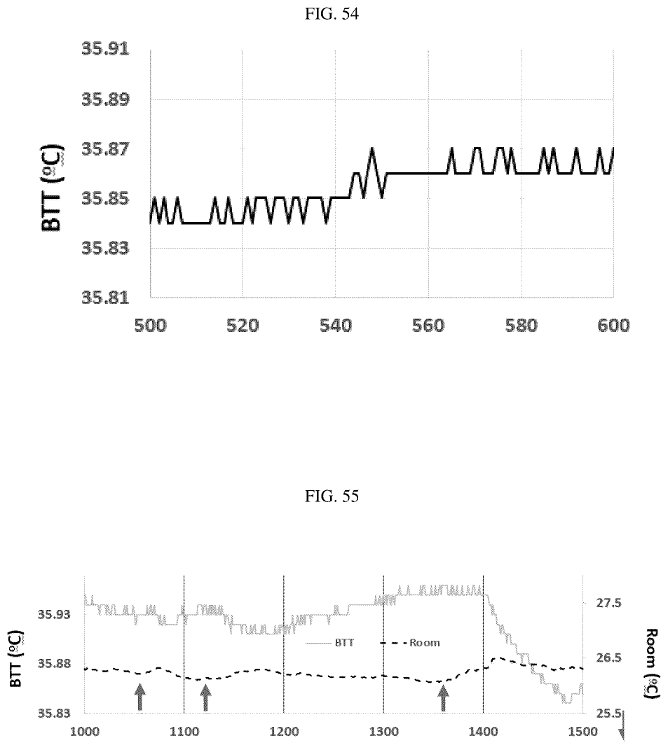

Data during room cooling and warming were obtained from volunteers who were testing the ability of ABTT wireless sensors in eyeglass frames in the presence of environmental disturbances, including changes in room temperature. While the changes in room temperature did not distort sensor values, they were found to cause substantial changes in thermoregulatory activity. In a healthy 32 year old male volunteer (who on repeat testing on a different day became pre-febrile-2), a 0.42° C. decline in room temperature over the cmyse of 600 sec, caused ABTT target area 10 temperature to increase by 0.11° C. See FIG. 53. As per the response to hand and arm immersion in cold water, this decline appeared to be a neurally mediated counterpart (from the periphery) to the findings in perfused hypothalamus of rabbits that activation of cold receptors on the hypothalamic surface initiated an increase in Symp. The pattern of narrow and broad impulses is shown for a 100 sec window in FIG. 54. The B/N incidence and occupancy ratios of 2/17 and 8/34 in a 100-sec window in FIG. 54 documented predominance of narrow impulses, thereby indicating a homeostatic “anti-hyperthermic” Psymp response to Symp-mediated increase in mean temperature.

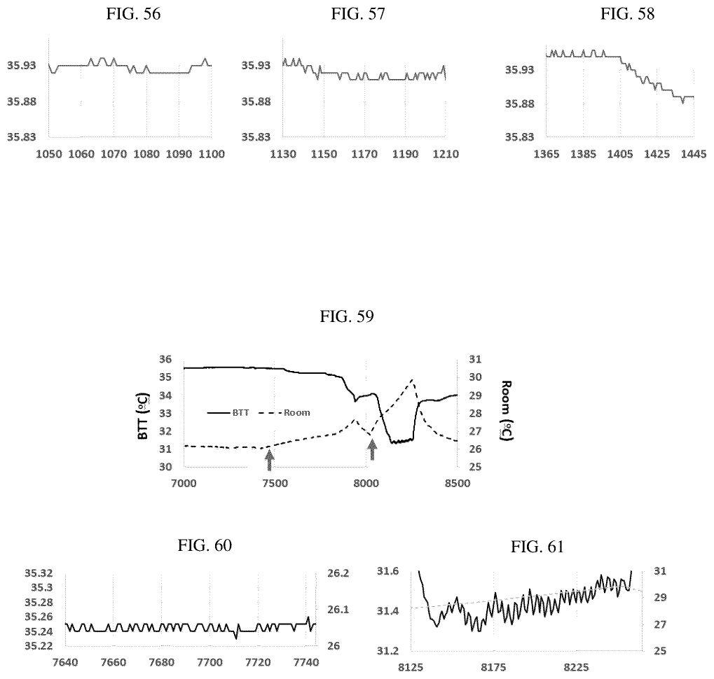

Two series of warmed room challenges were performed in the subject previously exposed to room cooling. The first series of challenges are shown in FIGS. 55-58. These challenges consisted of warming room temperature by 0.06° C., 0.13° C. and 0.20° C. Corresponding declines in ABTT target area 10 temperature were −0.01° C., −0.02° C. and −0.11C, at respective velocities of 0.04°·100 s⁻¹, 0.045°·100 s⁻¹ and 0.94°·100 s⁻¹. As shown in the graphs of FIGS. 56-58, imp&fluc were suggestive of Psymp activation, with B/N incidence ratios of 0/6, 3/10 and 0/10 and B/N occupancy ratios of 0/12,12/20 and 0/20. The findings thereby showed concurrent decrease in mean temperature and increase in relative Psymp impulses in response to the ambient warming. The highest increase in mean temperature during the third exposure was deemed potentially attributable to its being during the final and greatest of the three changes in room temperature, all of which were deemed small.

Approximately two hours later, during which time the subject was asleep, the subject underwent a series of two additional challenges shown in FIGS. 59-61. The challenges included warming room temperature by 1.32° and 2.82°. Corresponding declines in ABTT target area 10 temperature were −1.85° and −2.66°, at respective velocities of −0.404° C.·100 s⁻¹ and −2.375° C.·100 s⁻¹. The latter, in particular, was startling. The decreases in amplitude of −1.85° and −2.66° exceeded the means of that achieved during the entire engorgement period of males and female and the maximum change achieved during needle injection for a tattoo. The 0.404° C.·100 s⁻¹ decline during the first warming exceeded the mean of overall progressive engorgement (but not rapid pre-orgasm) velocity of males and females; it did not exceed that during needle injection for tattoo. Alternatively, the 2.375°·100 s⁻¹ decline in velocity during the second warming exceeded all of the other velocities. The graph in FIG. 60 shows that during a 100-second window at the onset of the first warming before the actual trough, the B/N ratios for incidence and occupancy were 4/12 and 16/24, consistent with the predominance of Psymp impulses.

The changes in the graph in FIG. 61 were unexpected. The seeming abrupt interruption of the decline in ABTT target area 10 temperature in FIG. 59 was accompanied by (perhaps secondary to) a previously unseen predominance of 4 to 9-sec impulses, with B/N incidence and occupancy ratios for 100 sec along the trough of 22/0 and 130/0. This data indicates that Symp can be activated to restrict Psymp-mediated suppression (hypothalamic hypothermia) when it reaches an extreme. This activation was previously demonstrated near the end of delta stage of sleep (and thus may be an important mechanism for precluding hypothermia during sleep). The differences between the first and second sessions in this subject may have been attributable to conversion to sleep (which, although it maintains autonomic activity, was deemed to have potentially augmented hypothermic response), greater increases in room temperature during the second session and the potential that the first session prompted the hypothalamus to be on alert in case there was another “hyperthermic threat.”

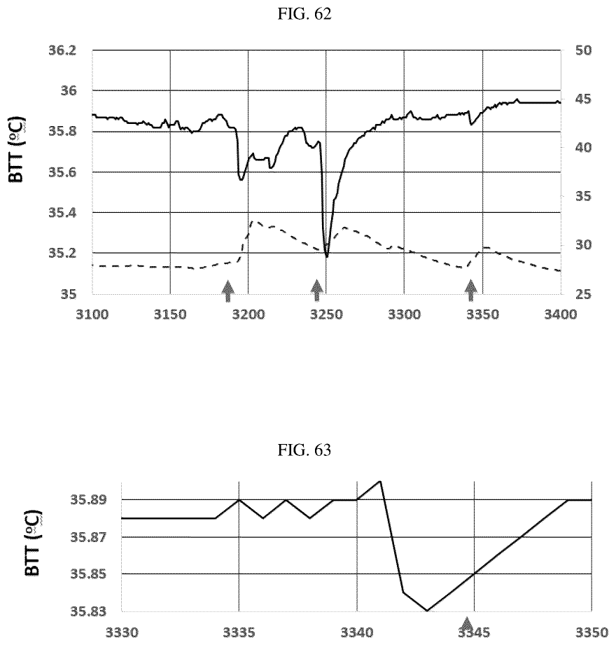

The previously unappreciated inconsistent consistency of thermoregulatory dynamics during room warming was also seen in the data from a second 32 year old healthy male volunteer, as shown in FIGS. 62 and 63. This awake subject underwent changes in room temperature more akin to the second series in the first subject. A series of three challenges consisted of warming room temperature by 4.08°, 2.3°, and 2.13° C. Corresponding declines in ABTT target area 10 temperature were −0.24°, −0.56°, and −0.16° C., at respective velocities of −4.0°·100 s⁻¹, −9.3°·100 s⁻¹, and −1.0°·100 s⁻¹. The amplitudes of ABTT target area 10 temperature decline were intermediate between those of the two sessions above. The velocities more closely resembled the faster velocities of the second session. One of the velocities during this third session (−9.3°·100 s⁻¹) far exceeded that seen in any other session, even though the accompanying change in room temperature was not extreme. The high velocities obscured impulses, if any. The graph of FIG. 63 shows a brief segment which identifies three 2 sec impulses prior to the third trough for B/N incidence and occupancy of 0/3 and 0/6.

The response of Psymp during what was deemed to be a potential hyperthermic threat (increased ambient temperature) was similar to that prior to other challenges. The range of change in amplitude encompassed the changes seen during anticipatory cooling prior to stressful mental effort (−0.09° C.) and at onset of hand and arm immersion in cold water (−0.13° C.). Likewise, for respective changes in velocity: −0.9°·100 s⁻¹ and −0.65°·100 s⁻¹.

Next is a discussion of pre-febrile thermodynamics during incipient infection: the key to unnmasking COVID-19 prior to signs and symptom. Access to graphs from three volunteers (the first two of which were cited above) who unknown to subject or staff were incubating influenza virus while evaluating BTT wearable sensors enabled the inventor to determine whether the autonomic thermodynamic activity documented during the aforementioned challenges likewise was identifiable during incipient viral disease. The inventor anticipated that ABTT target area 10 temperature would identify, and potentially characterize, the series of overlapping antigenic and immunologic processes which impact hypothalamic temperature.

Viewing the processes from the perspective of ABTT target area 10 temperature documentation of hypothalamus regulation of autonomic thermodynamics, the hypothalamus primarily is alerted to peripheral detection and immunologic response to viral invasion by transmission via visceral (and somatic) afferents from sites of activity and by initial activation of blood-borne immunologic compounds herein grouped together as “cytokines.” In this “cyto-autonomic” thermodynamic model, the former initiates a Psymp anti-thermic response as if responding to room warming. However, whereas this response normally would lower hypothalamic temperature and suppress Symp activity (leading to the low B/N ratio seen in Table 19), the progressive release of cytokines during immunologic and inflammatory responses limits Psymp activation, thereby inhibiting the inhibitor of Symp neuronal activity and inducing thermogenesis which progresses to stimulation of the HP axis, heat retention and fever.

To the extent possible with the inventor's preliminary data from asymptomatic infection, the inventor examined the hypothesis that, despite lack of signs or symptoms of a challenge (e.g., lack fever and lack of tachycardia, due to absence of Symp release), in the presence of incubating virus, the inventor could identify Symp, Psymp and cytokine expansion of the thermodynamic spectrum such that heretofore “invisible” antigen would become “visible” at a previously unseen thermoregulatory level during asymptomatic apparent euthermia.

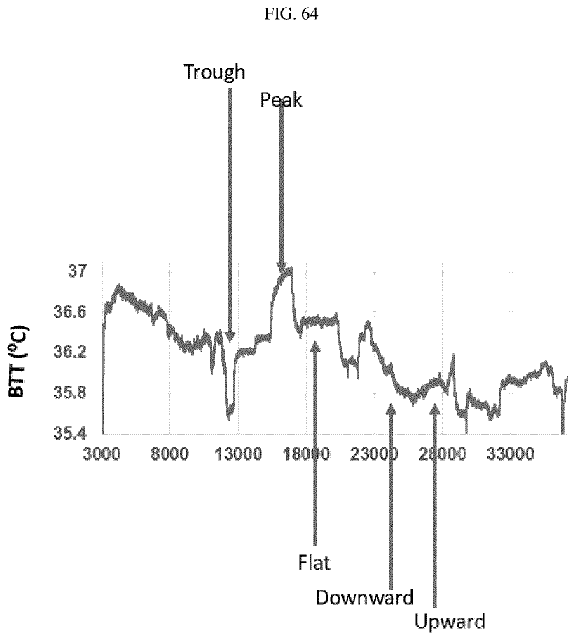

Proceeding sequentially through the cmyse of pre-febrile illness, the inventor began with the data from a pre-febrile-2 subject who was totally asymptomatic at the time of ABTT target area 10 temperature monitoring. As noted elsewhere herein, a comparison to a prior session in the same subject when he was healthy revealed distinctions between pre-febrile and healthy states. ABTT target area 10 temperature had reached 36.8° C., compared to a maximum of 35.96° C. when normal; even a 500 sec “flat” portion of pre-febrile-2 evidenced greater variability, thereby suggesting the potential for heretofore undefined changes within the thermal signal. Occasional spikes and precipitous troughs in the pre-febrile-2 graph were noted above; these spikes and precipitous troughs were identified as being critical features of virus-induced thermodynamics but on gross inspection did not delineate precipitation of the spike by increased cyto-autonomic activity. The inventor now sought to characterize heretofore unseen cyto-autonomic thermodynamics not only as the basis for but also in the absence of such intermittent quantal changes. Continuous monitoring consisted of two ˜30,000 sec recording periods separated by 3,600 sec. The first period, shown in FIG. 64, had an unusually wide range of thermodynamic patterns. The overall variability of the first period markedly exceeded the variability of its 500 sec “flat” segment described above. Mean (SD) and 5^{th}-95^{th} percentiles for the first period were 36.21° C. (0.6° C.) and 35.62° C.-36.82° C., respectively.

The inventor selected five sequential intervals that encompassed the gamut of grossly visible patterns of pre-febrile-2 variability. Each delineated a pattern of activity that had not been seen in prior challenges. Despite the absence of signs or symptoms of a disturbance or known presence of a challenge, ABTT target area 10 temperature evidenced virtually continuous activity throughout each of the 100-sec segments, regardless of whether there was apparent euthermia, upward and downward slopes, or peaks and troughs.

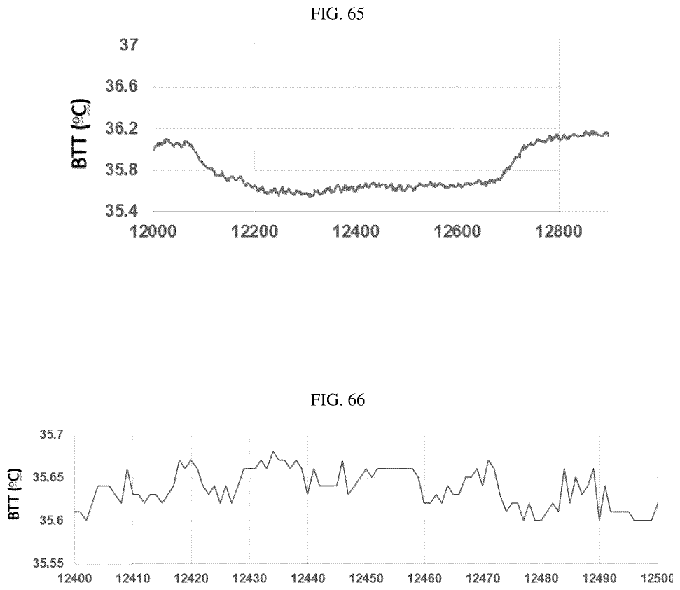

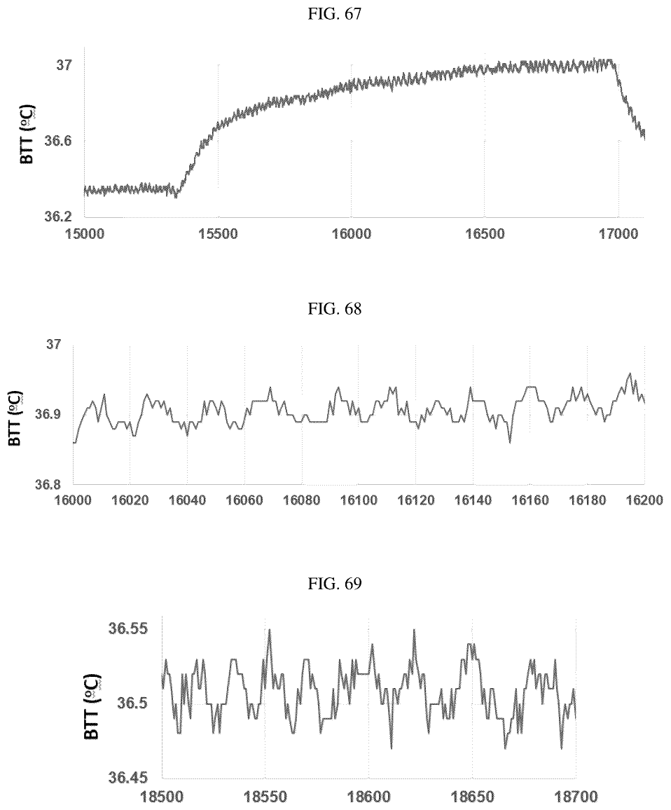

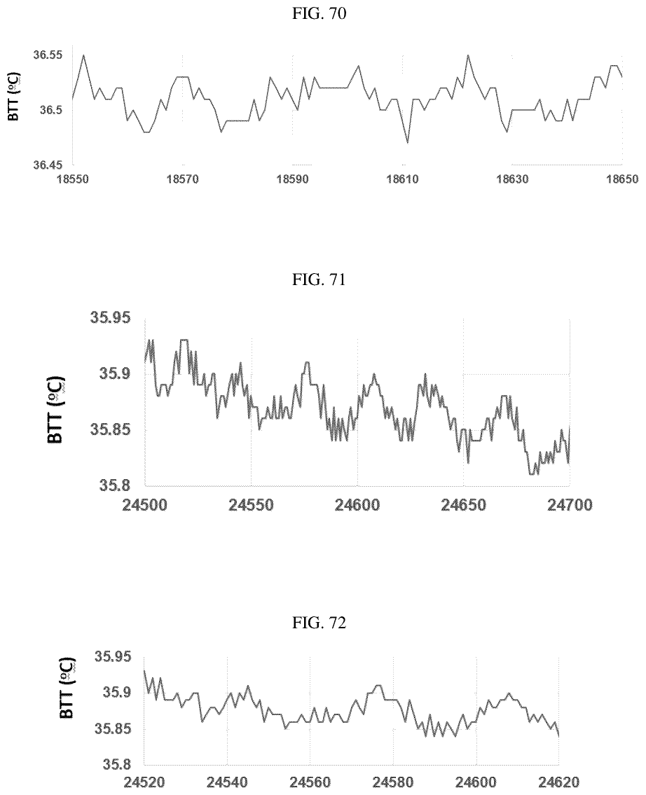

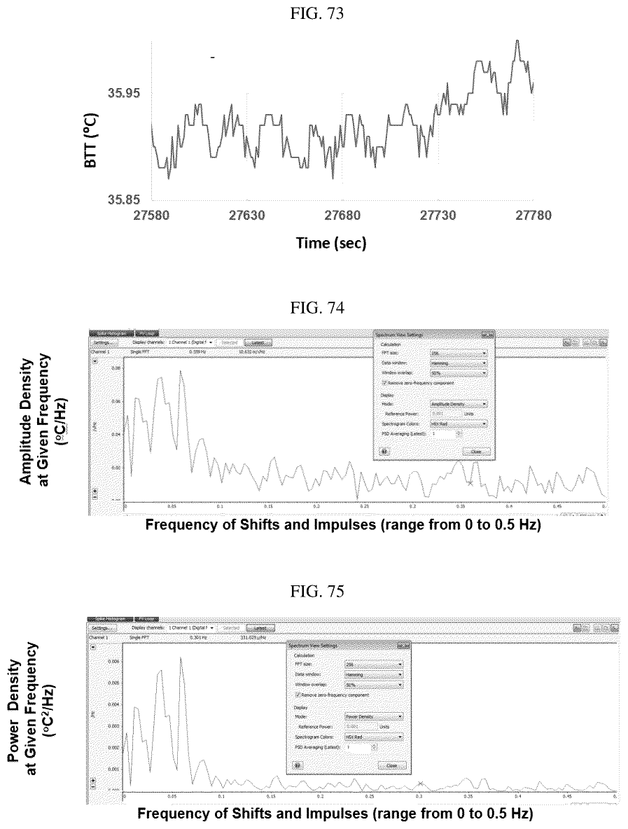

In each of five pre-febrile-2 windows, the cumulative values for ≤2 sec impulses and ≥10 sec activity were greater than that in either of two “healthy” 100-sec windows described above. Overall, the total window occupancy time (including impulses that overlapped) averaged 138.8 sec for the five pre-febrile-2 first period windows. This time was more than twice the average (53.5 sec) for the window occupancy time of the two intervals assessed when the subject was healthy. Remarkably, despite itself being without an apparent challenge, the total window occupancy time for each of the five pre-febrile-2 windows exceeded total window occupancy time during any of the challenges reported herein (corrected, when indicated, to 100-sec windows): 59.1 and 75.6 sec during engorgement and post-orgasm, respectively (mean of eight sessions); 70 sec during pauses between dye injection for tattoo (six sessions); 76 and 59 sec during stage 2 and REM sleep (n=1); 21, 91, and 45 sec during rise, plateau and recovery during stressful mental effort (n=1); 80 and 108 sec during initiation of and plateau during arm and hand immersion in cold water (n=1); 54 sec during room cooling, and, except for one session, was always less than 65 sec during room warming. The lone exception was unique abundance of Symp impulses at the nadir of profound Psymp-induced hypothermic trough as shown in FIG. 61. Further examination of the graphs revealed that it was the nature of the >10-sec changes, more so than their quantity, that distinguished the pre-febrile subject. Except for rare instances where signal assessment was complicated by a concurrent change in mean temperature as a consequence of an ongoing challenge, the B/N ratio of prior challenges clearly was based solely on imp&fluc wherein fluctuation was deemed to be a series of prolonged, rapidly recurrent and/or merged impulses that were of the same height and likewise returned to baseline. In contrast, FIGS. 65-73 document a series ≥10 sec shifts of baseline rather than activation of imp&fluc. Serial shifts were components of an oscillatory continuum around a new baseline, with peaks that exceeded the otherwise consistent height of impulses and fluctuations. Although shift height was greater than that of impulses and traditional fluctuations (which consistently have been the same in prior contexts), the ˜0.04°/9 sec rate of rise of the shifts in the example herein was less than that of adjacent impulses (˜0.02° C./1 sec).

The predominance of ≥10 sec shifts is seen in the tabulated data where >10-sec occupancy was at least 5× greater than 4-9 sec occupancy in each of the pre-febrile. Within the ≥10 sec band, the predominance of shifts as opposed to fluctuations was evidenced by their respective incidences in the five graphs (38 and 0, respectively). The findings were distinct from those of healthy subjects in the context of room warming, as shown in FIGS. 55-63; with the possible exception at the bottom of the Psymp-induced trough of FIG. 59, shifts consistent with potential cytokine release were not seen. The distinctions were reflected by the ≥10 sec/≤2 sec and ≥10 sec/4 to 9 sec ratios as well as the newly appreciated shift/fluc ratio.

FIGS. 74 and 75 show spectral displays with respect to amplitude density (in° C./Hz) and power density (in° C.²/Hz) of continuous ABTT target area 10 temperature signal during 500-sec flat phase of the first monitoring period of subject pre-febrile-2. In both spectra, continuous oscillatory signal identified cyclical nature of cytokine-induced shifts encompassing baseline as well as neurally derived imp&fluc. Predominant amplitude density is between 0.04 and 0.06 Hz, consistent with shifts in range of 20 to 25 sec width. Higher minimum values at these frequencies reflect higher baseline. Higher maximum values are based on cumulative magnitude of the oscillations at given frequency. Values in upper tracing (amplitude density) readily interchangeable with data as displayed in raw signals and thus easily applied for quantifying relationships among signals and over time. Lower channel included because of its common use for assessing cardiovascular signals and for its application above, as shown in FIG. 52, to assess thermoregulatory oscillatory activity during sleep. Comparison suggests that peak oscillations in subject with incipient fever greater than those during REM sleep.

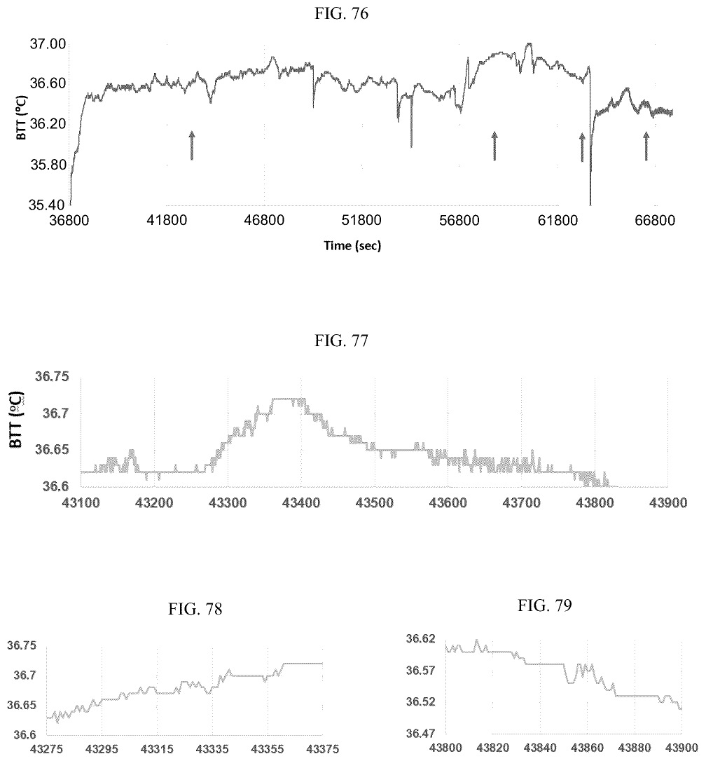

The shifts during each section of the first period of pre-febrile-2 were components of a continuum. The oscillatory nature of baseline variation, as opposed to isolated impulses, is shown by the spectral pattern of FIGS. 76-80, wherein oscillatory amplitude density over the 500 sec “flat” interval was maximal between 0.04 and 0.06 Hz (intervals between 16.6 and 25 sec). This amplitude far exceeded the amplitudes of the isolated impulses and fluctuations (which may be expressed with indices including relative amplitudes or power, percentage of total amplitude or power, and relationships between amplitude and baseline shift). Although they had consistent height, imp&fluc had varying widths and were separated by inconsistent distance along a nonoscillating baseline. (In addition to quantifying oscillatory amplitude and density within a given window and comparing cumulative totals, spectral domain analysis can be assessed serially in multiple displays and, as for cardiovascular waveforms, with joint time-frequency analyses. In addition, the spectral displays can provide a common platform for comparing central and peripheral changes. Signal comparisons may be extended to temperature signals at peripheral sites.

In the absence of sufficient Psymp modulation, as may be achieved by cytokine predominance (e.g., in more advanced disease) or sympathomimetic predominance (post-orgasm, pause after needle injection for tattoo), shifts may convert to a persistent rise (which does not return to baseline). Although the infection-induced response may resemble the change associated with the rise during a sympathomimetic challenge those challenges do not meet the combined criteria of: 1) Presence of shifts and accompanying changes in baseline; and 2) Depending on the stage of disease incipiency, either shift and Psymp impulses account for almost all window occupancy (since Symp impulses are suppressed by Psymp) or shift and Psymp impulses have reached cumulative level of ≥100 sec occupancy time (within 100 sec window). (As seen with more advanced disease below, a window may consist exclusively of cytokine activity, as Psymp impulses have disappeared). Stressful situations may induce release of cytokines; however, they are not associated with persistent release akin to that seen here in the context of incipient infection; and they are not likely to produce the persistent oscillatory pattern of FIGS. 65-73. Nor does sympathomimetic stress cause relative suppression of 4 to 9 sec impulses.

FIGS. 77-79 show the regions beginning at the 1^{st} arrow in FIG. 76, with continuous ABTT target area 10 temperature during >400 sec plateau early in during second period of pre-febrile-2 was indicative of extended impact of cytokine-induced shift. Lower tracings: upslope (100-sec window from 43275 to 43375 sec) and downslope (100 sec window from 43800 to 43900 sec) at the beginning and end of cytokine-induced rise.

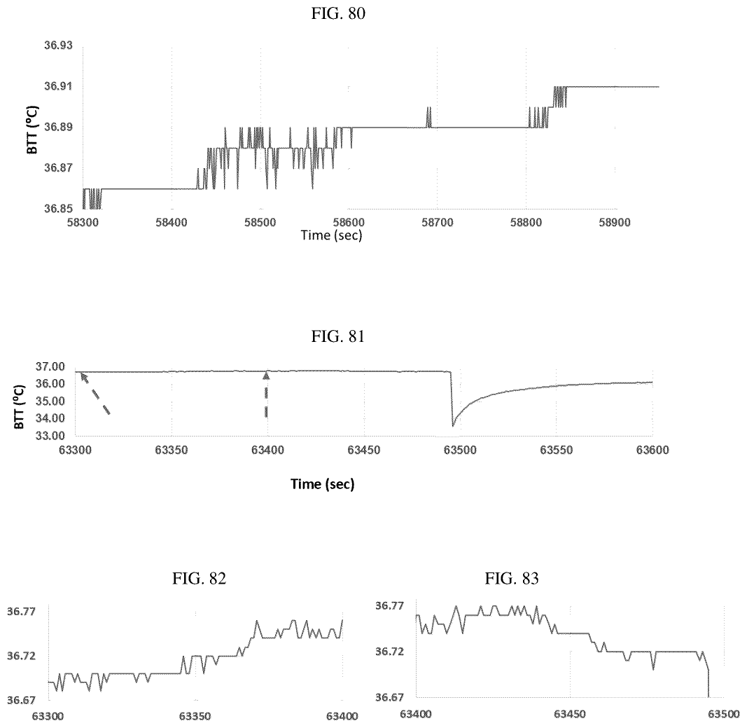

FIG. 80 shows the interval beginning at the 2^{nd} arrow in FIG. 76, a continuous 600-sec interval (58300 to 58900 sec) shows recurrent dominance of cytokines on ABTT target area 10 temperature signal. Psymp activity persists but appears in increasingly small clusters amidst predominance of prolonged (cytokine-induced) shifts.

FIGS. 81-83 begin at the 3^{rd} arrow of FIG. 76. There is a rise in ABTT target area 10 temperature until a major decline at 63450 sec. The predominance of Symp activity prior to the decline is shown in FIGS. 82 and 83 (as ˜100-sec shift). Midway through the graph of FIG. 83 (which began at the 2nd dashed arrow in FIG. 76) there is a gradual decline from 36.77° to 37.72° for ˜50 sec until there is a precipitous drop to 33.6. This drop is followed by rise to 36° C. and progressive return toward pre-trough values.

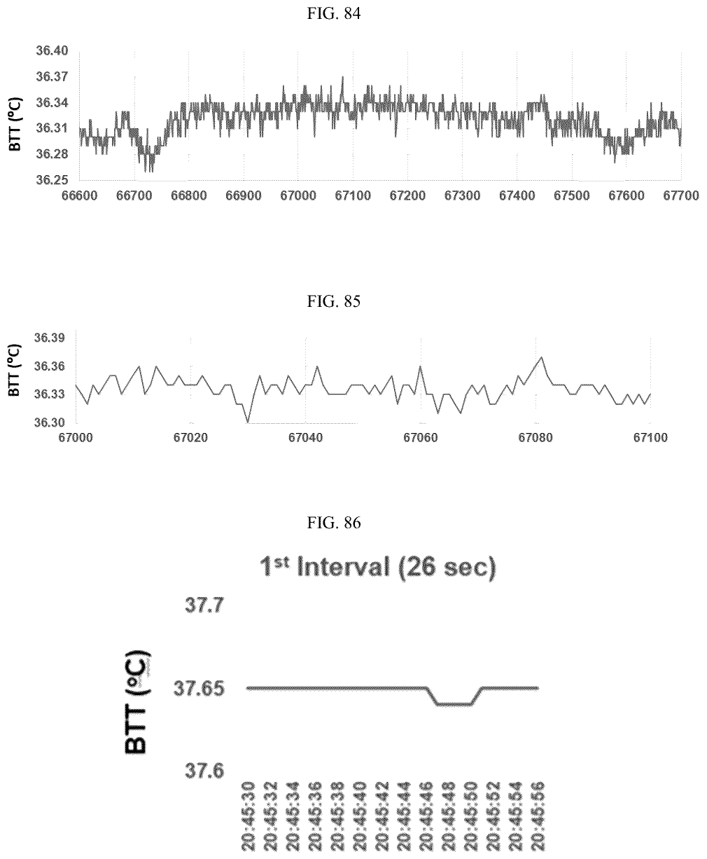

FIGS. 84 and 85 begin at the 4^{th} arrow in FIG. 76. Continuous ABTT target area 10 temperature near the end of the second period shows cytokine-induced shift persistent for approximately 850 sec (from ˜66750 to 67600 sec). FIG. 85 shows a graph of a 100-sec window from the middle of the ˜850 sec shift, documenting the predominance of Symp activity (despite apparent persistence of Psymp). This shift is consistent with attempts to modulate the rise in temperature as the setpoint rises toward fever, i.e., the pattern of activation and modulation repeats itself in the progression toward fever.

Comparison of periods 1 and 2 in FIGS. 64 and 76, respectively, documented that the impact of cytokines was even more predominant during the late portion of the second period of pre-febrile-2 testing. As opposed to waning of the progression toward fever between 25,000 and 33,000 sec of period 1, ABTT target area 10 temperature was maintained throughout period 2: peaks and oscillations were progressively longer and occupancy by Psymp impulses was decreased. Consistent with the reduced impact of Psymp activity, the second period of pre-febrile-2 monitoring had a higher mean temperature (36.57° C.) than the first period and a lower variability: SD of 0.36° C.; and 5^{th}-95^{th} percentiles of 36.34° C.-36.86° C. Moreover, the nature of occupancy changed dramatically. On gross inspection, it was apparent that, after ˜40,000 sec, there were multiple segments characterized by wide shifts indicative of cytokine dominance and attempts at temporary suppression (in the context of insufficient Psymp modulation).

The inventor examined progressive sections, identified by arrows in FIG. 76, throughout the second period. These progressive sections often required windows well beyond 100 sec, so as to capture the extent of cytokine-induced oscillatory changes in baseline and mean temperature. In each of the windows, there was predominance by cytokine-induced shifts such that they consistently occupied at least 80 sec of the window. Broad occupancy ranged from 100 to 195 sec within a 100 sec window; many shifts lasted 100 s of seconds. Narrow impulse occupancy was reduced, ranging from 0 to 40 sec, with regions of what appeared to be cytokine-induced Psymp silence. It became increasingly evident that the subject was transitioning from activated Symp/Psymp balance to cytokine/Psymp imbalance.

In the context of seemingly inadequate Psymp modulation of Symp activity, there were episodes of intense suppression, the most dramatic of which was at 63495 sec. The rate of rapid decline (3.15°/1 sec) was faster than the precipitous declines of −0.24°/6 sec and −0.56°/6 sec in response to the third series of room warming as shown in FIGS. 62 and 63. Overall, atop the initial rise(s) prior to the first measurement showing that BTT°-Core° difference already had reached 2.25° C. (shown above), the segments evidenced an initial 0.32° C. decrease, followed by an overall 0.32° C. increase in ABTT target area 10 temperature readings over the cmyse of 104 minutes of intermittent monitoring. This continued increase was accompanied by a paucity of narrow (≤2 sec) impulses (4↑,1↓) during the cumulative 135 seconds of BTT°. Shifts (as likely would be induced by cytokines) were underestimated in the short windows (wherein the given segment actually may have been part of a shift that was not identifiable within the window span); broad dominance nonetheless was evident as the B/N occupancy ratio was 50/10.

Not surprisingly, ABTT target area 10 temperature reached 37.99° C. during the fifth interval (while Core° was 37.2° C.). This difference was consistent with the BTT°-Core° discordance between measurements in the emergency room patients described above. In addition to higher mean temperature, pre-febrile-1 also had cyto-autonomic thermodynamic features suggestive of the later portion of pre-febrile-2, with a high ratio of shift/Psymp activity. Hence, when concurrent measurements of Core° are available, it may be helpful to view them (and hence BTT°-Core°) in concert with cyto-autonomic thermodynamics obtained from the BTT° signal.

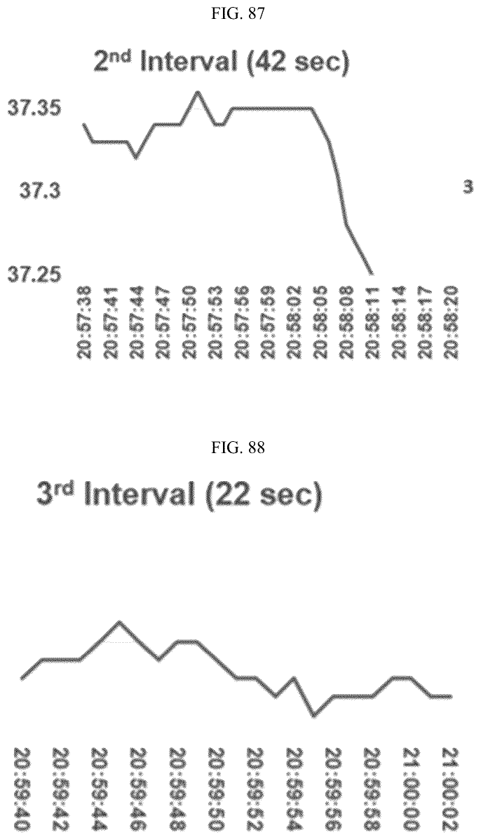

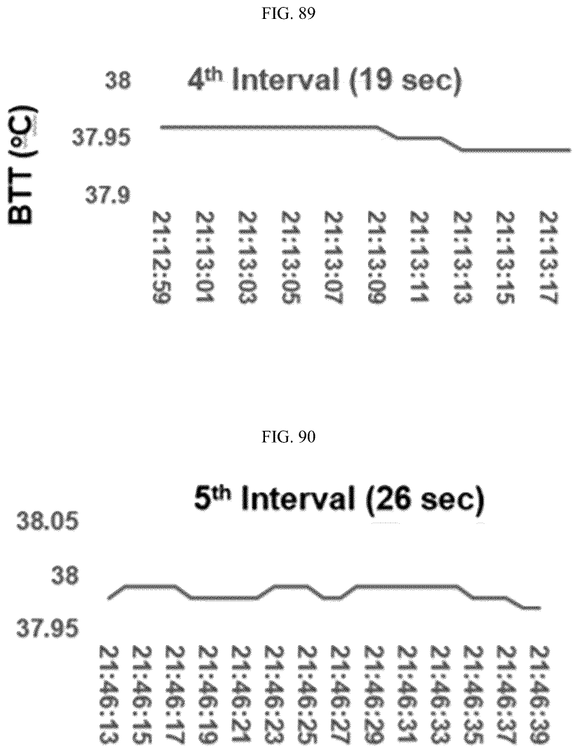

FIGS. 86-90 show five brief intervals of continuous ABTT target area 10 temperature in a volunteer (pre-febrile-1 described above) with fatigue who unknowingly was in incipient state of influenza, which became manifest one day later. The five segments, which are not continuous with each other, are arranged with 0.10 range along y-axis. The brief windows show paucity of narrow impulses, consistent with dominant impact of cytokines. The likelihood that each of these segments is atop a cytokine-induced shift is suggested not only by the paucity of Psymp but also by the overwhelming predominance of cytokine-induced shifts near the end of pre-febrile-2 as well as well as the variability of BTT° at the onset of different windows.

Thus, there are at least two patterns of cyto-autonomic thermodynamic activity in the individual with asymptomatic incubating infection (within the combined criteria noted above). Both of these are characterized by cytokine-induced shifts and changed autonomic activity. In the early phase, there is abundant Psymp activity in response to peripheral inflammation, such that ≤2 sec and ≥10 sec activity combine for otherwise unattained total occupancy. (Subjects who are earlier in the incubation than pre-febrile-2 may show an earlier rise in one of the cyto-autonomic thermodynamic (thermoregulatory) components, e.g., Psymp predominance before detectable impact of cytokines (addressed below). Alternatively, a disproportionate ≥10 sec/≤2 sec ratio may prove to be pathognomonic for later portions of the pre-febrile phase, i.e., activation of Symp-induced shifts without Psymp modulation.

The features of the pre-febrile subjects were in the absence of obvious challenge (although they also could be present during the febrile stage of illness). When indicated, a transient challenge or provocateur can be identified by questioning the subject and ruling out certain ongoing activities. Additionally, extended monitoring may be achieved during sleep. This monitoring may be especially valuable for detecting intermittent quantal changes, as well as patterns of impulses, fluctuations and shifts. While, based the inventor's findings above, the inventor does do not anticipate that prolonged monitoring of ABTT target area 10 temperature would be required to identify the presence of COVID-19 in its incipient states, overnight testing would provide a convenient setting for further delineation of patterns of activity and to identify changes including frequency and nature of fluctuations and changes in Psymp activity secondary to disease progression and impact of therapy. Wireless monitoring via a mobile app during sleep would enable widespread accrual of data not only for individual assessment but also for cumulative analysis at a central monitoring center in accordance with machine learning. Hence, the importance of the above confirmation that, under normal conditions, impulses associated with hypothalamic activity are maintained during sleep.

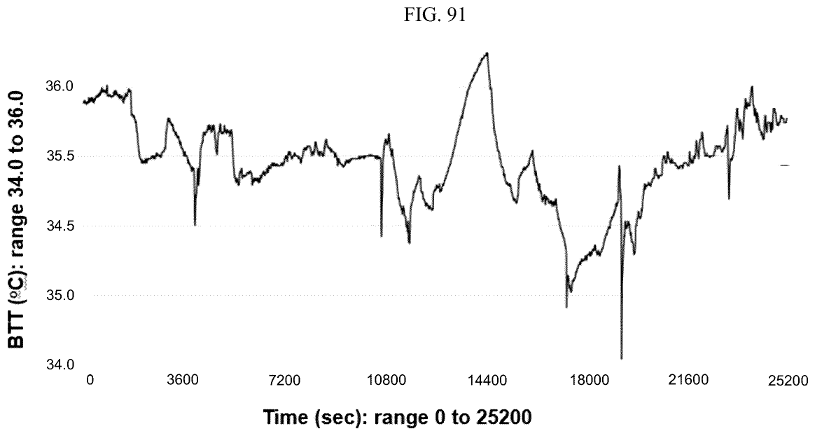

The archives which provided access to the findings of pre-brile-1 and -2 also provided access to a third unknowingly pre-influenza volunteer, an asymptomatic 32-year-old male (here called pre-febrile-3) who was continuously monitored while testing high resolution sensing headgear and data streaming during sleep. FIG. 91 shows a pre-febrile-3 graph from sleep onset until awakening that revealed the normal initial sleep pattern with gradual decrease of ABTT target area 10 temperature, as observed in the inventor's other sleep studies. However, there was a sudden Symp-driven surge of ABTT target area 10 temperature at approximately 4 hours. This surge had a velocity of 0.06° C.·100 s⁻¹, which is consistent with thermodynamics (0.07° C.·100 s⁻¹) of pre-febrile-2 (who was awake). Likewise, consistent with pre-febrile-2, predominantly Symp input was followed by predominantly Psymp tone, with sudden reduction of ABTT target area 10 temperature (−0.14° C.·100 s⁻¹), in agreement with Psymp thermodynamics during asymptomatic awake state (−0.13° C.·100 s⁻¹). Consistent with the inventor's documentation of persistent autonomic thermodynamics during sleep (above), these changes suggest the ability of the thermoregulatory center to maintain autonomic responsiveness to infection. Unfortunately, the data for this graph during equipment testing was not archived for subsequent detailed analysis. In the absence of a data file, we relied on inspection of the graphs and graphic enhancements thereof. This nonetheless showed an intriguing change over the cmyse of the study, consistent with the progressive changes in Psymp and cytokine activity during viral incubation. There are approximately fmy (cytokine-induced) shifts occupying most of the final 1000 seconds of the tracing.

Additional Impacts on the Battle Against COVID-19. In addition to providing heretofore unachieved identification of viral invasion during its incubating phase, continuous monitoring of the ABTT target area 10 temperature signal and its delineation of cyto-autonomic thermodynamics has alerted us to a feature of the autonomic response that may be contributing to “delayed” detections by screeners and care providers as well as “delayed” response by the host. The inventor's preliminary findings suggest that the hypothalamus initially may respond to the peripheral inflammation provoked by invading antigen akin to its response to surface warming, with predominance of Psymp impulses. By seeking to protect the host from perceived threat of hyperthermia, Psymp activation may be preventing rather, than responding to, Symp activation and inadvertently may be masking the early signs of viral proliferation and the immune response (e.g., tachycardia, fever). This may explain silent infiltration of (and shedding from) the oropharynx, where changes of local milieu in this heat-sensitive region access the hypothalamus via a rich network of visceral autonomic afferents. There likewise is input from the liver as it filters viral-immunologic complexes, which similarly may activate Psymp. In contrast to bacteria, viruses require host cells in which to multiply; depending on the sites of viral multiplication, this may promote selective afferent signaling.

The inventor's findings in the context of thermal challenges delineate the basis for these confounding impacts. Responses during hand and arm immersion in cold water and exposure to decreased room temperature identified Psymp modulation of the impact of these sympathomimetic challenges. Moreover, as noted above, exposure to increased peripheral temperature, e.g., room temperature as shown in FIGS. 55-63 and initial hand immersion in warm water, as shown in FIG. 36, induces seemingly unbridled activation of Psymp anti-hyperthermic mechanisms which we believe would substantially compromise an inflammatory response. If the hypothalamus interprets the local inflammatory responses to be manifestations of imminent hyperthermia, then, rather than allowing an unbridled sympatho-immunologic response to destroy the virus, it could bridle the response, thereby deferring an effective response until after the horse (or better, “herd” of viruses) is out of the barn (multiplying at highly infectious and potentially highly injurious rate within the cells).

In addition to the potential for Psymp activation to promote stealth of the virus, it may compromise health of the host, most notably by a virus with a long incubation period. The inventor's findings suggest that herein introduced normothermic analysis of ABTT target area 10 temperature can effectively address these confounders. Although a disturbance may be suggested by measurements of mean ABTT target area 10 temperature and its overall variability, this disturbance initially may be imperceptible, is not specific for infection, and does not identify the components of cyto-autonomic thermodynamics. Alternatively, viral invasion can be detected, characterized, and potentially diagnosed by monitoring cyto-autonomic thermodynamics in accordance with the inventor's herein proposed criteria which, in addition to delineating Psymp activity, also focus on the presence of cytokine-induced thermo-autonomic and thermo-immunologic shifts. From a therapeutic standpoint, delineation of cyto-autonomic thermodynamics may guide interventions that safely promote killing of virus while it is limited in number and not well-sequestered. It may also enable one to deliberately alter the cyto-autonomic thermoregulatory activity so as to activate a desirable sympatho-immunologic response, perhaps by challenges akin to those shown herein (e.g., room cooling, hand immersion in cold water). It similarly may enable indications for and timing of other interventions, including antivirals, administration of antibodies, vaccinations and of medications which activate (e.g., liposaccharides) or alter (e.g., hydroxychloroquine, interferon) the immunologic response, as addressed below. Alternatively, monitoring cyto-autonomic activity also may enable intervention when the inflammatory response becomes excessive (e.g., cytokine storm), as was shown to occur upon exposure of vaccinated animals (against SARS-Cov) to virus (Tseng et al., 2012), and upon re-exposure after recovery from dengue fever, and that may occur in response to high viral loads.

Thus, the inventor has a test that identifies a disturbance that appears to be specific for infection(s). The patterns delineated in the pre-febrile subjects analyzed herein were clearly distinct from those at rest, during sleep, pre- and post-orgasm, pre- and post-dye injection for a tattoo, stressful mental effort, immersion of hand and arm in cold water, exposure to room cooling and warming. Findings on different days confirmed that the pattern seen in pre-febrile-2 at approximately 48 hours prior to symptoms was attributable to the incipient infection and not a feature of the given subject: tracings from the same subject on different days at rest and during his participation in studies of room cooling and room warming lacked such a pattern. The findings also distinguished the difference between a pattern one day before symptomatic influenza and ≥2 days prior to symptomatic influenza, thereby delineating the progress of disease.

The inventor believes the assessment of continuous ABTT target area 10 temperature monitoring and more specifically cyto-autonomic assessment of the ABTT target area 10 temperature signal can be introduced immediately to identify individuals with a pattern suggestive of cyto-autonomic disturbance (viewing in the spectral- and/or time-domains as in examples shown herein). Unless the findings can be dismissed based on questioning or evidence of confounding ongoing activity (highly unlikely, thus far none found), these results should prompt infectious precautions pending precise diagnostic viral nucleic acid testing. Moreover, in view of the dependence of a virus on host cells for survival and proliferation, early quarantining and appropriate distancing may not only prevent multi-host viral spread but also community virus survival. Hence, benefits can be achieved immediately for patient and community. As presented in the discussion, the signatures delineated with cyto-autonomic thermodynamics that results in BATS_{AI} may provide a biophysical (nonchemical) brain-based diagnostics that may replace the need for invasive and uncomfortable swabs, or invasive blood testing, with results being immediately available to patient and doctor to promptly administer any needed therapy to preserve heath and prevent death.

Concurrently caregivers, providers and investigators, as well as governments, health organizations and industry can be identifying specific cyto-autonomic profiles. The inventor believes that, consistent with reports that immune-HPA axis interactions appear to be virus and phase specific, assessment of cyto-autonomic thermodynamics will provide thermal profiles and even signatures of COVID-19 and other infectious as well as noninfectious disorders; however, one first needs to obtain a database to define a given disorder (e.g., COVID-19) and multiple data bases to ensure specificity for the given disorder. Much of this can be obtained during initial implementation.

The findings obtained with ABTT target area 10 temperature cannot be achieved at other sites of temperature measurement. Although sharing a common parameter (temperature) with ABTT target area 10 temperature, they differ as a consequence of vascular transmission of the thermal signal to sites which introduce the features which confound traditional thermometry (e.g., varying dermal thickness and fat, distortion by feces, cerumen, recent ingestion of food or water, and susceptibility to inconsistent blood delivery) and highly reactive, unpredictable, and erratic vasomotor tone that characterizes regions outside the brain-eyelid tunnel as shown in numerous studies herein. It thus is not surprising that sites such as the forehead, sublingual and finger have grossly different temperature characteristics. As shown during sexual relations and receipt of a tattoo, they also respond differently to challenges (in large part the consequence of peripheral innervation) from ABTT target area 10 temperature. Nonetheless, when monitoring at such sites is present, comparisons to ABTT target area 10 temperature may provide insight in central vs peripheral activity.

From a novel therapeutic perspective, the ability to treat infection at inception via the means used by nature to fight infections, i.e., fever induction by the hypothalamus, albeit here noninfectious programmed fever, may prove to be a uniquely effective and safe therapeutic modality to achieve victory against COVID-19. History has shown previous victories by this matchless brain-based biological power: from the distant past by infectious fever treating syphilis to the present time with noninfectious programmed fever leading to restoration of neural function in neurodegenerative diseases, and Alzheimer's disease. The consistent restoration of neural function in neurodegenerative diseases by programmed fever provides the foundation for using this region of spectrum to prevent and treat COVID-19, since similar thermodynamics and high temperature treated a challenging infection (neurosyphilis). Thermodynamics through brain-based cyto-autonomic waves may thus open the path for a single biophysically-based method to defeat COVID-19, by not only detecting but also treating asymptomatic infection by SARS-COV-2.

From a practical standpoint, introduction of the proposed BTT brain-based monitoring may alter the maxim, “Take two aspirin and call in the morning.” Instead, the recommendation may be to withhold antipyretics (e.g., aspirin) which inhibit the sympatho-immunologic inflammatory response until signs and symptoms of disease become harmful or intolerable (e.g., high fever). Perhaps this would lead to an alternative maxim: “DON'T take two aspirin, stream to me ymy cyto-autonomic thermodynamics (i.e., BATS_{AI}) and call me in 5 minutes.”

While various embodiments of the disclosure have been shown and described, it is understood that these embodiments are not limited thereto. The embodiments may be changed, modified, and further applied by those skilled in the art. Therefore, these embodiments are not limited to the detail shown and described previously herein, but also include all such changes and modifications. It should also be understood that any part of series of parts of any embodiment can be used in another embodiment, and all of those combinations are within the scope of the disclosure.

1. A method of identifying Covid in a patient, the method comprising:
positioning a temperature sensor on a patient's Abreu Brain Thermal Tunnel (ABTT) terminus located between an eyebrow and an eye of the patient;
continuously reading a temperature output from the ABTT terminus over a predetermined period of time;
displaying a graph of the continuously read temperature output on a display; and
identifying a characteristic Covid signature from the displayed graph.

2. The method of claim 1,
wherein the characteristic Covid signature is identified by observing, during the predetermined period of time, a series of first temperature changes in a baseline temperature of the ABTT terminus that each occur over a first period of time B, and
observing a series of second changes in the temperature output that each occur over a second period of time N.

3. The method of claim 2,
wherein an amount of the first temperature changes is greater than an amount of the second temperature changes over the predetermined period of time, the predetermined period of time being greater than the first period of time and the second period of time.

4. The method of claim 2,
wherein the first period of time B is greater than the second period of time N.

5. The method of claim 2, wherein the first period of time B is ≥10 seconds, and the second period of time N is ≤2 seconds.

6. The method of claim 1, wherein the predetermined time is a period of 100 seconds or greater.

7. The method of claim 2, wherein the characteristic Covid signature is identified by detecting when a ratio of a number of first time periods to a number of second time periods B/N observed during the continuous period of time is greater than a ratio B/N obtained during a time when the patient does not have Covid.

8. The method of claim 7, wherein the B/N obtained during the time when the patient does not have Covid is less than 1.

9. The method of claim 4, wherein the first period of time B being greater than the second period of time N is an indication of cytokine-induced alteration of the temperature of a hypothalamus of the patient.

10. The method of claim 2, wherein the first temperature changes in the baseline temperature of the ABTT terminus during a first portion of the predetermined time are increases in baseline temperature of the ABTT terminus.

11. The method of claim 10, wherein the first temperature changes in the baseline temperature of the ABTT terminus during a second portion of the predetermined time are decreases in baseline temperature of the ABTT terminus.

12. The method of claim 11, wherein the first portion of the predetermined time occurs before the second portion of the predetermined time.

13. The method of claim 1, wherein during the continuous reading of the temperature output from the ABTT terminus, the temperature sensor is attached to a temperature module that is configured to be worn on a head and face of the patient.

14. The method of claim 13, wherein the temperature module further includes:
an ear monitoring module, comprising:
a wireless transmitter;
a wire; and
a receiver; and
an ear probe;
wherein the wireless transmitter is wirelessly connected to the receiver and the display, and the wire terminates in the ear probe.

15. The method of claim 14, wherein the temperature module further includes a frequency analyzer or spectrum analyzer to perform frequency/spectral analyses of the continuously read temperature.

16. A method of detecting influenza in a patient, the method comprising:
positioning a temperature sensor on a patient's Abreu Brain Thermal Tunnel (ABTT) terminus located between an eyebrow and an eye of the patient;
continuously reading a temperature output from the ABTT terminus over a predetermined period of time;
displaying a graph of the continuously read temperature output on a display; and
observing, during the predetermined period of time, a series of first temperature changes in a baseline temperature of the ABTT terminus that each occur over a first period of time B, and
observing a series of second changes in the temperature output that each occur over a second period of time N,
wherein an amount of the first temperature changes is greater than an amount of the second temperature changes over the predetermined period of time, the predetermined period of time being greater than the first period of time and the second period of time.

17. The method of claim 16,
wherein the first period of time B is greater than the second period of time N.

18. The method of claim 2, wherein the first period of time B is ≥10 seconds, and the second period of time N is ≤2 seconds.

19. The method of claim 1, wherein the predetermined time is a period of 100 seconds or greater.

20. The method of claim 2, wherein the influenza is detected when a ratio of a number of the first time periods to a number of the second time periods B/N observed during the continuous period of time is greater than a ratio B/N obtained during a time when the patient does not have influenza.
